(19) United States
(12) Patent Application Publication
(10) Pub. No.: US 2003/0104390 A1
(43) Pub. Date: Jun. 5, 2003

(54) Use of biosensors to diagnose plant diseases

(76) Inventors: Laurent Etienne, Kingersheim (FR); Evelyn Schurmann, Zeihen (CH); Peter Oroszlan, Basel (CH); Markus Ehrat, Magden (CH); Gert Ludwig Duveneck, Bad Krozingen (DE); Andres Binder, Steinmaur (CH)

Correspondence Address: William A. Teoli, Jr., Syngenta Crop Protection, Inc., Patent and Trademark Dept., 410 Swing Road, Greensboro, NC, 27409, US

(21) Appl. No.: 09/968,428

(22) Filed: Oct. 1, 2001

Related U.S. Application Data
(63) Continuation of application No. 09/297,782, filed on May 7, 1999, now abandoned.

(30) Foreign Application Priority Data
Nov. 11, 1996 (EP) 96810772.2

(51) Int. Cl. C12Q 1/68; C12Q 1/70; C12M 1/34

(52) U.S. Cl. 435/6; 435/5; 435/287.2

(57) ABSTRACT

The invention relates to a biosensor for the diagnosis of plant diseases, which is suitable for recognising plant diseases, as well as its use in the course of this process, as well as a sensor platform as a component of a biosensor for the diagnosis of plant diseases, whereby the biosensor as an analytical measuring unit consists of the sensor platform according to the invention, which may be modified and on which immobilised biochemical recognition elements are immobilised, whilst in close contact with an appropriate transducer arrangement. The said biochemical recognition elements are structures which are specific for the plant pathogens to be evaluated, and therefore allow individual detection of these plant pathogens to be carried out in the course of the diagnostic process according to the invention.

The invention relates to a biosensor suitable for recognising plant diseases, as well as its use in the course of a diagnostic process.

Hereinafter, the specific recognition and quantification of plant pathogens are summarised under the expression “diagnosis”.

Biosensors are measuring instruments whose primary signal is produced by a biochemical reaction. The analytical measuring instrument consists of an immobilised biological material in close contact with an appropriate transducer arrangement. The transducer converts the biochemical signal into a quantifiable electric signal. (Gronow, 1984, Trends Biochem. Sci. 9, 336-340). The biosensor membrane recognises analytes on a molecular level, while the transducer detects the electrochemical, thermal, piezoelectric or optical changes at its surface. Sensors may be divided into the following groups according to signal recognition:

1. Electrochemical sensors

2. Piezoelectric sensors

3. Calorimetric sensors

4. Optical sensors

Electrochemical sensors are described in Wilson, G. S. 1987 in Biosensors: Fundamentals and Applications; (Turner, A. P. F., Karube I. & Wilson G. S., Eds.) pp 165-179, Oxford University Press, Oxford).

Calorimetric sensors are described in Danielsson, B. & Mosbach, K., 1987, in Biosensors: Fundamentals and Applications; (Turner, A. P. F., Karube I. & Wilson G. S., Eds.) pp 575-595, Oxford University Press, Oxford).

Piezoelectric sensors are described in Luong et al. TIBTECH 6, 310-316 (1988).

Detection is based on optical sensors, for example the measurement of change in colour, reflection, refraction index, fluorescence or chemoluminescence. Optical sensors take measurements either directly or indirectly; here, either the optical properties are changed by means of a reaction between the biological component and the analyte or a dye is integrated into the reaction and its depth changes through the reaction between the biocomponent and the analyte. Surface plasmon spectroscopy is an example of a direct measuring method. (Hall, E. A. H. (1986) Enzyme Microb. Technol. 8. 651-658). In optical biosensorics, the method of “internal total reflection spectroscopy” has increased in importance. (Robinson G. A. (1991) Biosensors & Bioelectronics 6, 183-191).

If a light wave is coupled into a planar waveguide which is surrounded by—media of a lower refractive index, it is confined by total reflection to the boundaries of the waveguiding layer. In the simplest instance, a planar waveguide consists of a three-layer system: substrate, waveguiding layer, superstrate (or sample to be investigated), whereby the waveguiding layer has the highest refractive index. Additional intermediate layers may further improve the action of the planar waveguide.

In that arrangement, a fraction of the electromagnetic energy enters the media of lower refractive index. This portion is described as an evanescent (=decaying) field. The strength of the evanescent field is greatly dependent on the thickness of the waveguiding layer itself, as well as on the ratio of the refractive indices of the waveguiding layer and the media surrounding it. In the case of thin waveguides, i.e. layer thicknesses that are the same as or smaller than the wavelength that is to be guided, discrete modes of the guided light can be distinguished.

Using an evanescent field for example, it is possible to excite luminescence in media of relatively low refractive index, and to do so only in the immediate vicinity of the waveguiding region. This principle is called evanescent luminescence excitation.

For analytical purposes, evanescent luminescence excitation is of great interest, since excitation is restricted to the immediate vicinity of the waveguiding layer.

The need for early identification of plant pathogens (A. Binder, L. Etienne, J. Beck, J. Speich & J. Youd, 1995. Practical value of crop disease diagnostic techniques. In: Hewitt et al (eds.) A vital role for fungicides in cereal production, SCI & BCPC Proceedings, UK, 231-238) has increased due to the need for judicious usage of pesticides in plant protection. Additional domains are interested in characterising the phytosanitary condition of seeds, plant material or the harvested plants. Of the numerous plant pathogens that are important in the diagnosis of plant diseases, notable ones are fungi, bacteria, viruses, viroids and phytoplasma. Which test method is used depends on the type of pathogen and the plant substrate to be examined. One method used originally to examine plant diseases was the visual evaluation of symptoms. Further examinations were normally carried out in the laboratories using microscopes or by isolating pathogens on artificial nutrients. Until a short time ago, improved examination methods were based on electron microscopy. However, electron microscopy is very time-consuming and therefore routine examinations cannot be carried out on a larger scale. A great advance was made in the development of serological examination methods based on immunological methods (F. M. Dewey & R. A. Priestley (1994): A monoclonal Antibody-based for the Detection of the Eyespot Pathogen of Cereals Pseudocercosporella herpotricoides. In Modern assays for Plant Pathogenic Fungi CAB international, 9-15) and a few disadvantages of the above-described methods could thus be eliminated.

Serological methods that are used in crop protection and are based on the ELISA techniques are described in an overview by 1. Barker (1996) (Serological methods in crop protection. In

Diagnostics in Crop Protection, BCPC Proceedings, 65, 13-22). Considerable progress has been made in the last 3 years in the development of testing methods based on DNA technology (RFPL, PCR, etc.). (J. D. Janse: (1995) New methods of diagnosis in plant pathology—perspectives and pitfalls. Bulletin OEPP/EPPO 25, 5-17).

An alternative analytical process to the ELISA technique, based on the use of fibre-optic evanescence field bioaffinity sensors, is described by P. Oroszlan et al. (Automated Optical Sensing System for Biochemical Assays: a Challenge for ELISA? Analytical Methods and Instrumentations, Vol. 1, No. 1, 43-51).

An overview over the use of these sensors on different bioaffinity systems is given by G. Duveneck in Proceedings SPIE, volume 2631, pp 14-28 (1996). The potential improvement in detection limits of bioaffinity sensors, based on the excitation of luminescence in the evanescent field of a waveguide through the use of thin-film metal oxide waveguides as transducers, is described in WO 33197 and in WO 33198.

Disadvantages of the above-mentioned processes are normally: high costs, since inter alia the platforms cannot be regenerated, too long analysis times due to complicated sample preparation work, purification steps for working up the plant extracts and too few samples being processed, since normally only one pathogen is examined at a time.

There is thus a need to develop a process which allows several samples of plant material to be examined for one or more plant pathogens in a parallel manner, i.e. simultaneously or directly after one another, without additional purification steps, and in addition enables the plant pathogens to be analysed and quantified early, in a highly sensitive manner, without time-consuming purification steps for the plant extract, and with a high number of samples.

Within the context of the present invention, it has now surprisingly been found that biosensors may be used in plant diagnostics for the early recognition of plant diseases, whereby the plant material to be examined can be used directly in the form of plant extracts without prior processing, in the course of the diagnostic process according to the invention. The use of biosensors in plant diagnostics results in the fact that from now on plant extracts can be examined with high sensitivity, more quickly, cheaper, in a fully automated manner and with a higher number of samples than was possible with prior-known processes. These biosensors may be employed both in the laboratory and directly in the field, and can be regenerated.

According to the use of the expression in this application, biosensors are measuring instruments whose primary signal is produced by a reaction with biological or biochemical analyte molecules. According to the definition used here, biosensors consist of chemical or biochemical recognition elements, immobilised on a so-called transducer which, as a consequence of the reaction with the biological analytical molecules, creates a change in state which can be converted into a quantifiable electronic signal. The transducer is generally a solid material. In the following, the expressions “transducer” and “sensor platform” are used synonymously. The chemical or biochemical recognition elements recognise analyte molecules on a molecular level; contact of the recognition elements with the transducer enables for example an electrochemical, piezoelectric, calorimetric or optical effect to take place as a consequence of the reaction with the analytes, and this effect can be subsequently converted into an electronic signal. Depending on the principle of the signal being produced, the following groups of sensors may be classified without restricting their general application and without regarding this as a complete list:

1. Electrochemical sensors

2. Piezoelectric sensors

3. Calorimetric sensors

4. Optical sensors

In principle, all of the above-mentioned biosensors, for example electrochemical sensors, piezoelectric sensors, calorimetric sensors or optical sensors, are suitable for the usage according to the invention in the course of the process according to the invention. Especially suitable, and therefore preferred in the context of this invention is the use of biosensors with sensor platforms, since these enable several sample solutions to be analysed with high sensitivity. Washing or purification steps between the individual measurements can be omitted, so that a high number of samples may pass through per unit of time. This is of great significance especially for routine examinations or for evaluations in the course of genetic engineering.

It has also surprisingly been found that, in a simple manner, a sensor platform may be produced on the basis of at least two separate regions on a common substrate, which is suitable for the parallel detection of the same or different analytes to diagnose one or more plant diseases.

Apart from examining several sample solutions simultaneously, one sample solution can also be examined for several analytes contained therein, simultaneously or in succession, on one sensor platform. This is particularly advantageous for examination of plant extracts, which can be carried out in a particularly rapid and economical manner.

A further advantage of the use of the sensor platform is that the individual separate regions may be addressed selectively either chemically or fluidically.

Preference is given to a sensor platform on the surface of which one or more specific binding partners are immobilised as chemical or biochemical recognition elements for one or more, same or different plant pathogens to be evaluated.

Especially preferred in the context of the invention are optical biosensors with a sensor platform, which are produced on the basis of one, preferably at least two planar, separate, inorganic, dielectric, waveguiding regions on a common substrate, and are suitable for the parallel evanescent excitation and detection of luminescence of the same or different analytes in order to diagnose plant diseases. These separate waveguiding regions may each contain one or more grating couplers.

If several sample solutions are analysed at the same time, the separate waveguiding regions prevent any cross-talking of luminescence signals from different samples. With this process, high selectivity and a low error rate are attained.

Through the separation of waveguiding regions, it is also possible to further increase selectivity and sensitivity with the well-directed usage of light sources of different wave lengths.

A further advantage of the use of the sensor platform in an optical biosensor for diagnosing plant diseases is that the individual separate waveguiding regions may be selectively addressed not only chemically or fluidically, but also optically.

Preference is given in the context of the present invention to the use of a sensor platform to diagnose plant diseases, which consists of planar, physically or optically separate waveguiding regions, in which only one or few modes are guided. They are notable for especially high sensitivity with the smallest possible construction. Normally, this sensitivity is not obtained by multimodal waveguides of planar construction.

Coupling-in of the excitation light may take place for example using lenses, prisms, gratings or directly into the end face of the waveguiding layer.

Coupling-in and, where appropriate, coupling-out using gratings is normally simpler and more efficient than with lenses or prisms, so that the intensity of the coupled-in light wave is similarly greater, which, in conjunction with low degree of attenuation of the guided lightwave, contributes towards very high sensitivity of this arrangement.

Sensitivity may be further augmented by using as strong an evanescent field as possible. This offers the possibility of determining even the smallest amounts of luminescent material on the surface of the waveguiding layer.

One object of the present invention thus relates to a sensor platform as a component of a biosensor, which is especially suitable for diagnosing plant diseases. The said biosensor, which is similarly a constituent of the present invention, essentially comprises a measuring instrument which contains as a component the sensor platform according to the invention. The said sensor platform may be modified and normally contains immobilised, plant pathogen-specific, biochemical recognition elements which are in close contact with a suitable transducer arrangement. The said biochemical recognition elements are structures which are specific for the plant pathogens to be evaluated and therefore enable individual detection of these plant pathogens to be made within the course of the diagnosis process according to the invention.

The said plant pathogens are preferably those selected from the group of fungi, bacteria, viruses, viroids and phytoplasms, but especially fungi, selected from the sub-divisions of Mastigomycotina, Zycomycotina, Ascomycotina, Basidiomycotina or Deuteromycotina; bacteria selected from the group Agrobacterium, Spiroplasma, Clavibacter, Erwinia, Pseudomonas, Xanthomonas or Xylella; as well as viruses selected from the group carla virus, clostero virus, cucumo virus, luteo virus, nepo virus, potex virus, poty virus or tobamo virus or from the group phytoplasmosis.

Especially preferred in the course of this invention are sensor platforms which bear chemical or biochemical recognition elements that are specific for phytopathogenic fungi selected from the group of the genera Aphanomyces, Pythium, Phytophthora, Plasmopara, Bremia, Pseudoperonospora or Peronospora; Podosphaera, Sphaerotheca, Erysiphe, Uncinula, Nectria, Giberella (Fusarium), Glomerella, Claviceps, Scierotinia, Cochliobolus, Leptosphaeria (Septoria), Pyrenophora, Venturia, Guignardia Uromyces, Puccinia, Hemileia, Ustilago, Tilletia, as well as Typhula.

A further object of the invention relates to a sensor platform for diagnosing plant diseases, which consists of one or more, but especially two separate regions on a common substrate.

Also included in the invention is a sensor platform whose signal activation is based on the transduction principle of electrochemical, piezoelectric, calorimetric or optical transduction mechanism.

A sensor platform whose signal activation is based on an optical transduction mechanism is preferred.

In a particular embodiment of the present invention, there is a sensor platform whose signal activation is based on the change in resonance conditions to produce a surface plasmon resonance by means of interaction between one or more, identical or different plant pathogens to be evaluated with one or more specific binding partners as chemical or biochemical recognition elements, which are immobilised on the sensor platform.

Especially preferred is a sensor platform whose signal activation is based on interaction between one or more, identical or different plant pathogens to be evaluated with one or more specific binding partners as chemical or biochemical recognition elements in the evanescent field of a waveguide.

Particularly preferred is a sensor platform whose signal activation is based on the effective refractive index in the evanescent field of a wave guided in an optical waveguide through interaction of one or more, identical or different plant pathogens to be evaluated with one or more specific binding partners as chemical or biochemical recognition elements, which are immobilised on the sensor platform.

A further object of the invention relates to a sensor platform whose signal activation is based on the change in the coupling angle of a grating coupler through interaction between one or more, identical or different plant pathogens to be evaluated with one or more specific binding partners as chemical or biochemical recognition elements, which are immobilised on the sensor platform.

Preference is given to a sensor platform whose signal activation is based on the change in a luminescence signal through interaction between one or more, identical or different plant pathogens to be evaluated with one or more specific binding partners as chemical or biochemical recognition elements, which are immobilised on the sensor platform.

Especially preferred is a sensor platform based on a planar, dielectric optical waveguide, but especially a sensor platform based on a planar, dielectric optical waveguide, with which luminescence may be evanescently excited and detected.

Most particularly preferred in the context of this invention is a sensor platform based on at least two planar, separate, inorganic dielectric waveguiding regions on a common substrate.

A specific embodiment of the present invention relates to a sensor platform for the diagnosis of plant diseases, which consists of a continuous transparent substrate and a transparent, planar, inorganic, dielectric waveguiding layer, which is characterised in that

a) the transparent, inorganic, dielectric waveguiding layer is subdivided at least in the measuring region into at least 2 waveguiding regions, such that the effective refractive index in the regions in which the wave is guided is greater than in the surrounding regions, or such that the subdivision of the waveguiding layer is formed by a material on the surface that absorbs the coupled-in light;

b) the waveguiding regions are each provided with or have a common coupling-in grating, so that the direction of propagation of the wave vector is maintained after coupling-in, and

c) where appropriate, the waveguiding regions are each provided with or have a common coupling-out grating.

Similarly included in the present invention is a biosensor for the diagnosis of plant diseases, which contains a sensor platform according to the invention and an appropriate transducer arrangement.

In addition, the invention relates to processes for diagnosing plant diseases in plant material and also in soil or air samples, using the biosensor according to the invention or the sensor platform according to the invention.

A further object of the invention relates to the use of the sensor platform according to the invention or the biosensor according to the invention in analytical processes for the diagnosis of plant diseases.

The present invention relates primarily to a sensor platform as a component of a biosensor, which is especially suitable for the diagnosis of plant diseases. The sensor platform according to the invention may consist of both one region and two separate regions.

In the present invention, the purpose of the separate waveguiding regions is to provide one sensor platform for the simultaneous detection of evanescently excited luminescence of one or more analytes.

The terms measuring section and measuring region are used synonymously in the context of the present invention.

The separate waveguiding regions may have any geometric form. This effectively depends on the structure of the whole apparatus in which the sensor platform is installed. Examples of geometric forms are lines, strips, rectangles, circles, ellipses, cross-hatches, rhombi, honeycombs or irregular mosaics. The divisions between the individual waveguiding regions essentially run in a straight line. At the ends, they may taper for example, and they may be broader or narrower overall than the measuring region.

The waveguiding regions are preferably arranged in the form of separate strips, rectangles, circles, ellipses, cross-hatches.

The waveguiding regions are most preferably arranged in the form of parallel strips. The waveguiding regions are most preferably in the form of parallel strips less than 5 mm apart. A further preferred embodiment is obtained if the waveguiding regions are arranged in the form of parallel strips which are joined at one or both ends, whereby the direction of propagation of the wave vector does not change after the coupling-in.

In a further advantageous embodiment, the strips are joined together at one end, while the other end is open, whereby the direction of propagation of the wave vector does not change after the coupling-in.

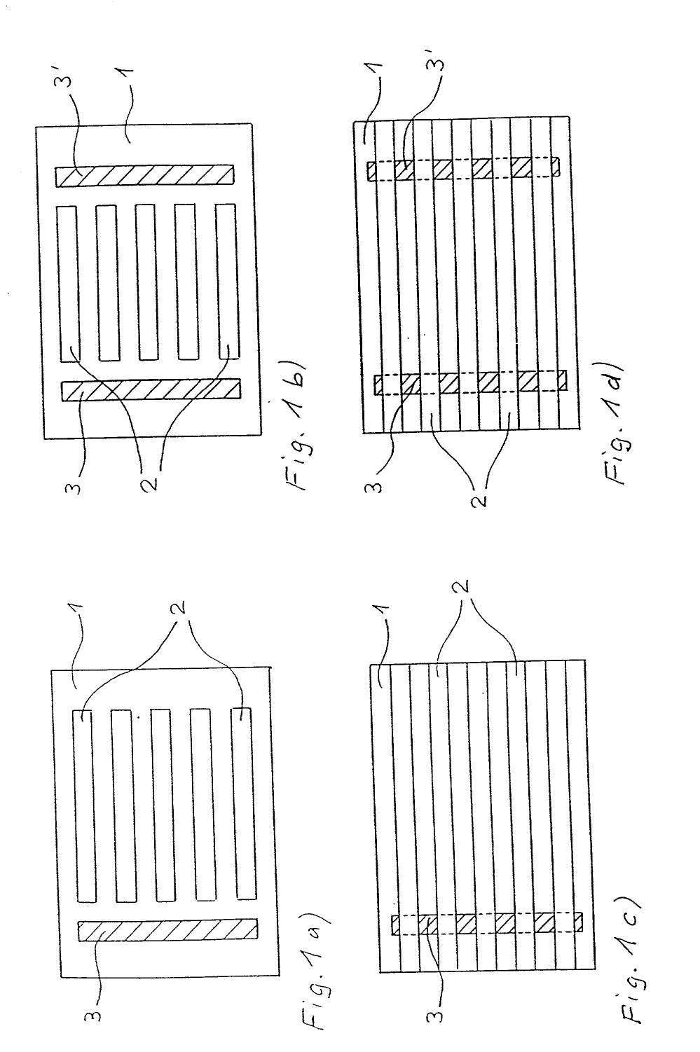

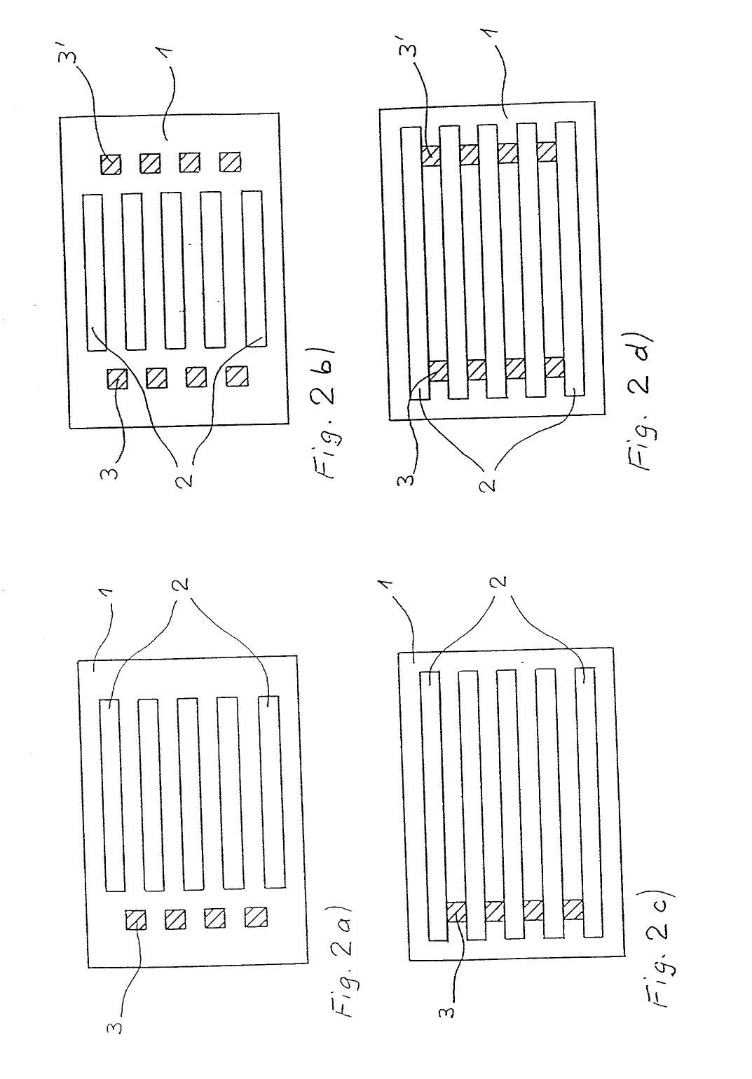

FIGS. 1a to 1d and 2a to 2d illustrate a few further possible arrangements. The reference numerals show:

1 the waveguiding layer which has been applied to a substrate;

2 the divisions which are either formed by an absorbing material on the surface of the waveguiding layer, or by a reduction in the effective refractive index in the plane of the layer, which is achieved most simply by means of an air gap in place of the waveguiding layer;

3, 3′ the coupling-in and coupling-out gratings.

In FIG. 1a, the waveguiding regions (=measuring regions) are broken up by dividing regions. These dividing regions do not come into contact with the coupling element.

In the case of FIG. 1b, coupling-in and coupling-out gratings are jointly available to all measuring regions. There is no contact with the dividing regions.

In FIG. 1c, the dividing regions extend beyond the coupling element. Coupling-in is however unaffected by these in the waveguiding regions.

FIG. 1d contains two grating couplers and otherwise corresponds to FIG. 1c.

FIGS. 2a to 2d show an arrangement in which the gratings couplers are not continuous, but an individual grating is assigned to each waveguiding region.

The physically or optically separate waveguiding regions may be produced using known processes. There are two possible basic processes. For example, a) the layers may be constructed from the start with physical separation in an vapour deposition method using masks, or b) a continuous layer is produced and this is subsequently structured using appropriate methods. One example of process a) is the vapour deposition of the inorganic waveguiding material, whereby a suitably constructed mask covers up part of the sensor platform. Such masks are known from the production of integrated circuits. Here, the masks should be in direct contact with the sensor platform. Positive and negative masks may be used.

It is also possible to apply a suspension of the inorganic waveguiding material to the sensor platform by means of a suitably constructed mask, and to produce the waveguiding layer by the sol-gel technique.

In this way, separate waveguiding regions are produced, whereby the division is created most simply by an air gap. However, this gap may also subsequently be filled with different material having a lower refractive index than that of the waveguiding layer. If division into several waveguiding regions is effected in this way, the difference in the effective refractive indices between the waveguiding region and the adjacent material is preferably more than 0.2, most preferably more than 0.6 units.

One example of process b) is the vapour deposition of an inorganic waveguiding material to form a continuous layer, which is subsequently subdivided into individual waveguiding regions by means of mechanical scoring, treatment with laser material, lithographic processes or plasma processes.

Vapour deposition normally takes place under vacuum conditions. Plasma deposition is similarly possible.

Special mention should be made of treatment with pulsed excimer and solid state lasers or continuous gas lasers. In the case of pulsed high-energy lasers, structuring may be effected over a large area through a mask. With continuously operating lasers, normally the focused beam is guided over the waveguiding layer to be structured, or the waveguiding layer moves relative to the beam.

The lithographic processes may be etching techniques, as employed in the production of printed circuit boards or microelectronic components. These processes allow an extraordinarily large number of geometric patterns to be produced and a fineness of structures ranging from micrometers to sub-micrometers.

What is important for all ablative operations is that the waveguiding layer is completely or partially removed, but the sensor platform is not completely divided. Any intermediate layers that are optionally present may similarly be completely or partially removed.

In a modified variant b) of the process, a continuous layer of an inorganic waveguiding material is applied first of all, and in a second step, using an absorbing material which interrupts the waveguiding, a structure is applied to this layer so that the waveguiding regions are divided by absorbing and thus non waveguiding regions.

The absorbent materials concerned may be inorganic materials such as metals with a high optical absorption coefficient, e.g. gold, silver, chromium, nickel or organic compounds, e.g. dyed and pigmented polymers. These materials may be applied to the waveguiding layer as continuous layers, or in the case of metals, in the form of aqueous colloidal solutions. Various methods may be chosen for this.

Deposition processes for structuring, which are carried out under vacuum conditions, have already been mentioned above.

Colloidal materials in water or organic solvents, for example gold in water, may similarly be employed for the structuring of waveguiding regions.

The deposition of colloidal gold onto surfaces by spontaneous assembly has been described for example by R. Griffith et al., Science 1995, 267, 1629-1632. Here, for example, physically or fluidically separate laminar part streams of a colloidal gold solution can be allowed to flow over the waveguiding layer, whereby the gold particles are deposited e.g. in the form of strips. The surface is dried, and separate, waveguiding regions according to the invention are obtained. The deposited gold colloids must have a minimum size of 10 to 15 nm for the desired absorption to occur. It is preferred if they are 15 to 35 nm in diameter.

Colloidal gold may also be deposited by stamping the surface. Stamping of dissolved organic materials is described by Whitesides as so-called ‘microcontact printing’ and has been used for structuring gold surfaces with liquid alkanethiols (J. L. Wilbur et al., Adv. Mater. 1994, 6, 600-604; Y. Xia and G. M. Whitesides, J. Am. Chem. Soc. 1995,117, 3274-3275). For example, colloidal gold solution can be drawn up into an elastomeric stamp having the desired structuring pattern, and the structuring pattern can be transferred to the waveguiding surface by applying the stamp.

Processes which operate with organic solvents or water are very flexible and quick to use. They enable waveguide structuring to take place directly before carrying out a luminescence assay.

Where appropriate, the surface of the waveguiding layer has to be modified prior to colloidal deposition of for example gold, so that good adhesion results between the colloid particles and the modified surface. Adhesion may be achieved by means of hydrophobic interaction, van der Waals forces, dipole-dipole interaction, simple electrostatic interaction or covalent binding. The interaction may be produced by functionalisation of the colloids and/or the surface of the waveguiding layer.

An appropriate method of modifying the surface and achieving adhesion is for example silanisation, as described in Advances in Colloid and Interface Science 6, L. Boksányi, O. Liardon and E. Kováts, (1976) 95-137. Such silanisation is also used to improve the adhesion of recognition elements in affinity sensing. Mercapto-terminated silane, for example (mercaptomethyl)dimethylethoxysilane, is especially suitable for the adhesion of gold by creating a covalent sulphur-gold bond.

Another modification of process b) is that, in a second step, the same inorganic material is applied in the form of a structure to the continuous layer of an inorganic waveguiding material, so that an increase in the effective refractive index is achieved by increasing the layer thickness, and thus the propagation of lightwave mode is concentrated in the resultant measuring regions. Such ‘slab waveguides’ and processes for the production thereof are described by H. P. Zappe in ‘Introduction to Semiconductor Integrated Optics’, Artech House Inc., 1995.

The width of the strip of waveguiding layers is preferably 5 micrometers to 5 millimetres, most preferably 50 micrometers to 1 millimetre.

If the width of the waveguiding regions is reduced too greatly, the available sensor region is also reduced. The strip width and required sensor region are conveniently matched to one another.

The size and width of the individual waveguiding regions may be varied within a wide range and basically depend on the purpose of use and the structure of the system as a whole.

The individual waveguiding regions, when formed as strips, preferably have a length of 0.5 to 50 mm, most preferably 1 to 20 mm and most preferably 2 to 10 mm.

The number of strips on the sensor platform is preferably 2 to 1000, most preferably-2 to 100.

The individual waveguiding regions may be arranged for example as strips on the substrate in two or more groups, each respectively having at least two strips, thus forming a multiple detection region.

The great practical advantage of multiple detection regions of this construction is that, between successive multianalyte measurements, the sensor platform does not have to be cleaned or replaced, but only displaced relative to the excitation unit, fluidics unit and detection unit.

A further advantage is that such multiple detection regions are economically more favourable to produce. A very substantial advantage is that the very time-consuming and cost-intensive division into individual sensor platforms may be dispensed with.

Each multiple detection region preferably consists of 2 to 50, most preferably 2 to 20 separate waveguiding regions.

There are preferably 2 to 100, most preferably 5 to 50 multiple detection regions on the sensor platform.

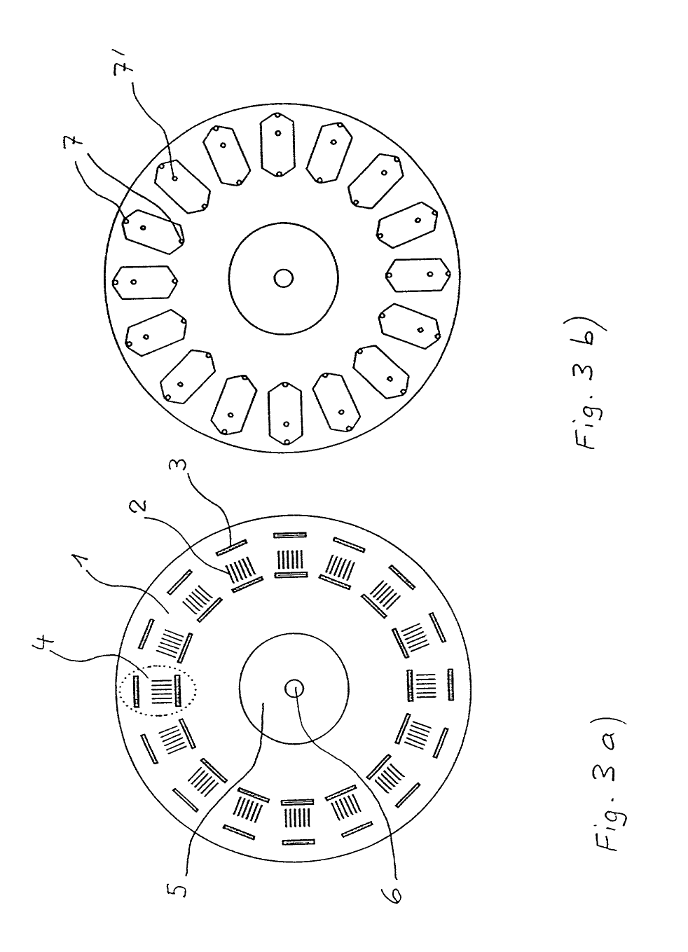

FIGS. 3a and 3b show a possible arrangement of a sensor platform with several multiple detection regions, in which the substrate has the shape of a disc and may be produced by press moulding in a similar way to current compact discs. The overall arrangement may consist of a disc-shaped sensor platform with several multiple detection regions and a fluidics disc, which contains the fluidics supply lines and the actual cell spaces. The two parts are joined, e.g. adhered, and form one unit.

The cell spaces in the form of wells may however also be preformed on the disc-shaped sensor platform. An embodiment of this type is then covered by a planar lid.

Reference numerals 1 to 3 have the significances indicated above, 4 indicates an entire multiple detection region, 5 signifies the substrate and 6 illustrates a central cut-out portion which can hold an axle, so that the individual multiple detection regions 4 can be rotated under excitation and detection optics. 7 and 7′ signify inlet and outlet apertures for the solutions required in the course of the assay, which are normally brought into contact, by means of a tnroughflow cell having at least two openings, with the recognition elements that are immobilised on the waveguiding regions.

The multiple detection regions may also be arranged on concentric circles. The spacing between the individual multiple detection regions may for example be such that, rotation through an angle between 5 and 20 degrees brings a new multiple detection region under the excitation and detection optics.

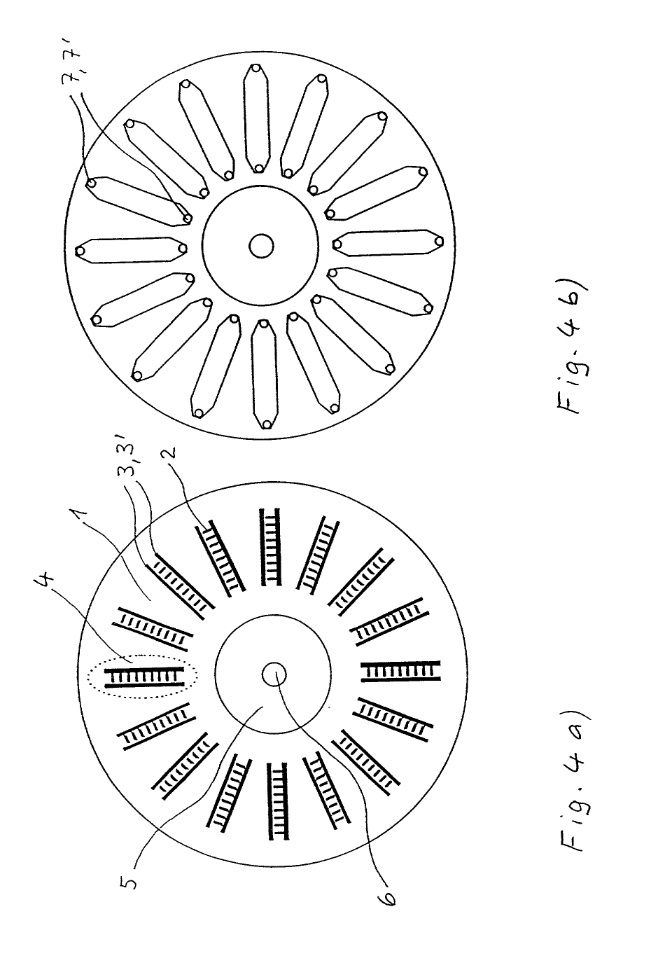

FIGS. 4a and b show an analogous construction of the sensor platform on a disc, with the difference that, in comparison with FIG. 3, the individual multiple detection regions 4 are arranged radially instead of tangentially, which leads to improved utilisation of the surface area.

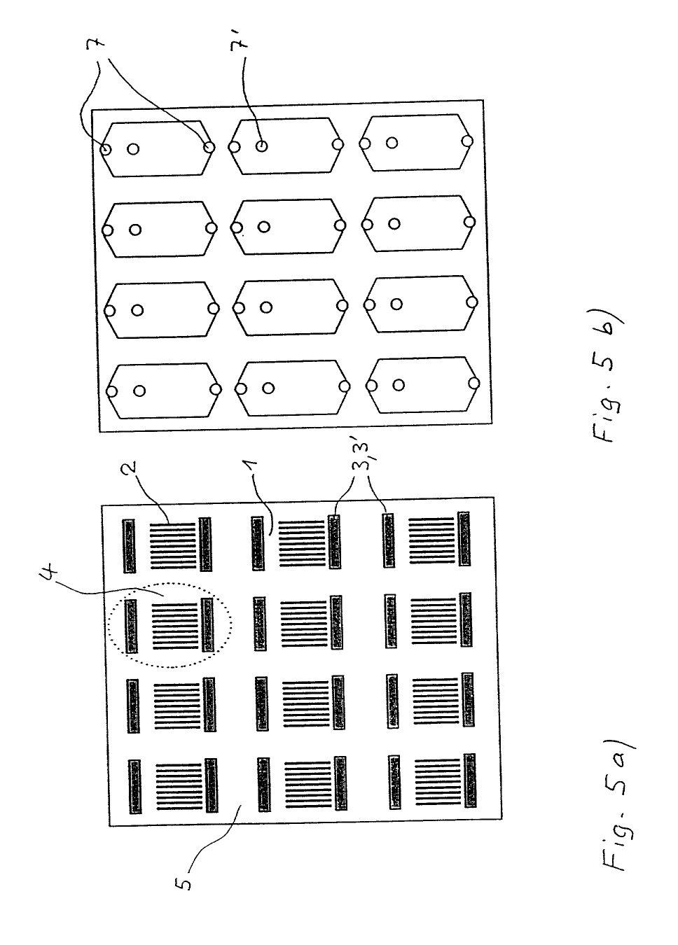

A further arrangement is illustrated in FIGS. 5a and 5b. The individual multiple detection regions 4 are arranged in the form of a rectangular cross-hatch pattern. However, the multiple detection regions may also be arranged as individual images in a film strip.

This film strip may be present as a planar element or may be rolled up.

The individual multiple detection regions may be transported under excitation and detection optics in a manner analogous to a film.

The preferences indicated for the separate waveguiding regions also apply to the multiple detection regions.

A sensor platform within the context of this invention is a self-supporting element which may be shaped as a strip, a plate, a round disc or any other geometric form. It is basically planar. The chosen geometric form is uncritical per se and may depend on the structure of the apparatus as a whole in which the sensor platform is installed. However, it may also be used as a independent element, physically separate from a source of excitation light and from the optoelectronic detection system. Arrangements that allow substantial miniaturisation are preferred.

Miniaturised systems are known for example from environmental analytics. These miniaturised systems are user-friendly and may also be used directly in the field.

The substrate may be for example glass of all kinds or quartz. Glass is preferably used, as this has the lowest possible optical refractive index and the lowest possible degree of intrinsic luminescence, and it allows the simplest possible optical machining to be carried out, such as etching, grinding and polishing. The substrate is preferably transparent, at least at the excitation and emission wavelengths. The microscopic roughness of the substrate should be as low as possible.

Transparent thermoplastic plastics may also be used as substrates, as are described for example in EP-A-0 533 074.

The substrates may be covered with a thin layer, which has a refractive index lower than or equal to the substrate and is no thicker than 0.01 mm. This layer may serve to prevent the interference of fluorescence excitation in the substrate and also to avoid superficial roughness of the substrate, and it may consist of a thermoplastic, a thermally crosslinkable or a structurally crosslinked plastics or also of inorganic materials such as SiO₂.

Where an intermediate layer is present, whose refractive index is lower than that of the waveguiding layer and whose layer thickness considerably exceeds the penetration depth of the evanescent field (i.e. in general>>100 nm), transparency of only this intermediate layer at excitation and emission wavelength is sufficient, if the excitation light beams in from the upper side of the sensor platform. In this case, the substrate may also be absorbent.

Especially preferred substrates are glass, quartz or a transparent thermoplastic plastics. Glass is preferred in particular.

Especially preferred substrates of transparent thermoplastic are polycarbonate, polyimide or polymethyl methacrylate.

It is preferable for the refractive index for all waveguiding layers to be the same, that is, all waveguiding layers preferably consist of the same material.

The refractive index of the waveguiding layers must be greater than that of the substrate and any optional intermediate layers used. The planar, transparent, waveguiding layer preferably consists of a material with a refractive index greater than 2.

The materials in question may be for example inorganic materials, especially inorganic metal oxides such as TiO₂, ZnO, Nb₂O₅, Ta₂O₅, HfO₂, or ZrO₂.

Ta₂O₅ and TiO₂ are preferred.

The thickness of the waveguiding layers is preferably 40 to 1000 nm, more preferably 40 to 300 nm, most preferably 40 to 160 nm.

In a preferred embodiment, the thickness of the waveguiding layers is the same.

The modulation depth of the gratings is preferably 3 to 60 nm, most preferably 3 to 30 nm.

The ratio of modulation depth to the thickness of the layers is preferably equal to or less than 0.5 and most preferably equal to or less than 0.2

The gratings for coupling in the excitation light or for coupling out the backcoupled luminescence light are formed as optical diffraction gratings, preferably as relief gratings. The relief structure may have various forms. Suitable forms are for example sinusoidal, rectangular or saw-toothed structures. Processes for producing such gratings are known.

Photolithographic or holographic processes and etching techniques are primarily used to produce them, as described for example in Chemical, Biochemical and Environmental Fiber Sensors V. Proc. SPIE, Vol 2068, 313-325, 1994. For organic substrates, moulding or stamping processes may also be employed.

The grating structure may be produced on the substrate and afterwards transferred to the waveguiding layer in which the grating structure is then reproduced, or the grating is produced in the waveguiding layer itself.

The grating period may be 200 to 1000 nm, whereby the grating advantageously has only one periodicity, i.e. it is monodiffractive. The grating period selected is preferably one that allows the excitation light to be coupled in the first diffraction order.

The modulation depths of the gratings are preferably of the same magnitude.

The gratings preferably have a bar to space ratio of 0.5-2. By bar to space ratio is understood for example the ratio of the width of the bars to the width of the spaces in the case of a rectangular grating.

The gratings may serve both to couple excitation light into the individual waveguiding layers and to couple out luminescence light backcoupled into the waveguiding layers.

In order to examine different luminescent samples, it may be expedient for all or part of the coupling-in or coupling-out gratings to have different grating constants.

In a preferred embodiment, the grating constants for all gratings are the same.

If some of the gratings are used for coupling in and some for coupling out the light, then the grating constant of the coupling-in grating(s) is preferably different from the grating constant of the coupling-out grating(s).

The grating distance is preferably B≦3·X_{1/e}, whereby X_{1/e} indicates the length at which the initial intensity I₀ of the guided beam has fallen to I₀/e.

One preferred group of embodiments of the sensor platform is characterised in that the transparent, planar, inorganic dielectric waveguiding regions on the sensor platform are divided from each other at least along the measuring section by a jump in refractive index of at least 0.6, and each region has one or two separate grating couplers or all regions together have one or two common grating couplers, whereby the transparent, planar, inorganic dielectric waveguiding regions have a thickness of 40 to 160 nm, the modulation depth of the gratings is 3 to 60 nm and the ratio of modulation depth to thickness is equal to or less than 0.5.

The jump in refractive index of 0.6 or more is most simply achieved whereby the waveguiding layer is divided completely and contains an air gap or, during measurement, optionally contains water.

The waveguiding regions preferably guide only 1 to 3 modes, and they are most preferably monomodal waveguides.

A further subject of the invention is a modified sensor platform for the diagnosis of plant diseases, which is characterised in that one or more specific binding partners are immobilised on the surface of the waveguiding regions as chemical or biochemical recognition elements for one or more, identical or different analytes.

In the course of this invention, a modified sensor platform is preferred, on the surface of which binding partners are immobilised as chemical or biochemical recognition elements, which are specific for the plant pathogens or properties of pathogens (e.g. fungicide resistance, virulence) to be determined and thus enable selective recognition of said pathogens to take place.

The biochemical recognition elements are in particular binding partners which are specific for indicator substances which are characteristic of the plant pathogens to be determined.

Specific binding partners which may function as chemical or biochemical recognition elements may be in particular antibodies, antigens, binding proteins A, binding proteins G, receptors, ligands, oligonucleotides, single strand RNA, single strand DNA, avidin, biotin, enzymes, enzyme substrates, enzyme cofactors, enzyme inhibitors, lectins or carbohydrates.

The plant pathogens which may be detected in the course of the diagnostic process according to the invention may be all plant pathogens from which said specific recognition elements can be isolated, but especially pathogens selected from the group comprising fungi, bacteria, viruses, viroids and phytoplasms.

Fungal pathogens may be taken from the classification of fungi according to Ainsworth (1971, Dictionary of the fungi, 6. ed. Comm. Mycol. Inst. Kew.) and Ainsworth, Sparrow, Sussman (1973, The fungi. Vol. IV A, IV B, Academic Press—New York, San Francisco, London).

Preferred target organisms among the fungal organisms are to be found within the division Myxomycota or Eumycota, and relate in particular to fungal pathogens from the subdivisions of Mastigomycotina, Zycomycotina, Ascomycotina, Basidiomycotina or Deuteromycotina; Especially preferred are the fungal pathogens of the subdivision Mastigomycotina, selected from the group of the genus Aphanomyces, Pythium, Phytophthora, Plasmopara, Bremia, Pseudoperonospora or Peronospora.

Furthermore, those that are especially preferred are fungal pathogens of the subdivision Acomycotina selected from the group of the genera Podosphaera, Sphaerotheca, Erysiphe, Uncinula, Nectria, Giberella (Fusarium), Glomerella, Claviceps, Sclerotinia, Cochliobolus, Leptosphaeria (Septoria), Pyrenophora, Venturia, Guignardia.

Furthermore, those that are especially preferred are fungal pathogens of the subdivision Basidiomycotina selected from the group of the genera Uromyces, Puccinia, Hemileia, Ustilago, Tilletia, Typhula.

Furthermore, those that are especially preferred are fungal pathogens of the subdivision Deuteromycotina selected from the group of the genera Rhizoctonia, Sclerotium, Verticillium, Botrytis, Pseudocercosporella, Pyricularia, Penicillium, Aspergillus, Rynchosporium, Cladosponum, Alternaria, Cercospora, Fusarium, Phoma, Ascochyta, Colletotrichum. Especially preferred are fungal pathogens selected from the group of the genus of Plasmodiophora, Spongospora, Polymyxa.

Especially preferred target organisms in the course of this invention are Septoria nodorum or Septoria tritici.

Within the bacteria group, the genera Agrobacterium, Spiroplasma, Clavibacter, Erwinia, Pseudomonas, Xanthomonas or Xylella are especially notable. These contain a number of plant pathogens.

Plant-pathogenic viruses are to be found in particular within the groups carla virus, clostero virus, cucumber mosaic virus, luteo virus, nepo virus, potex virus, poty virus or tobacco mosaic virus.

Preferred representatives of the phytoplasmoses which may be mentioned are for example representatives of proliferation disease and rubber wood disease of the apple.

Suitable chemical or biochemical recognition elements which are immobilised on the surface of the sensor platform according to the invention are in particular binding partners which are specific for indicator substances that are characteristic for the plant pathogens to be determined.

Specific binding partners which may function as chemical or biochemical recognition elements may be in particular antibodies, antigens, binding proteins A, binding proteins G, receptors, ligands, oligonucleotides, single strand RNA, single strand DNA, avidin, biotin, enzymes, enzyme substrates, enzyme cofactors, enzyme inhibitors, lectins or carbohydrates.

Especially preferred as specific binding partners in the context of this invention are DNA sequences from the Internal Transcribed Spacer (ITS) of the ribosomal RNA gene region, which are specific for various species and strains of Septoria, Pseudocercosporella, Fusarium and Mycosphorella and are described in WO 95129260.

Especially preferred as specific binding partners in the context of this invention are also plant pathogen-specific antibodies or antigens, but especially antigens, which may be obtained from the fungal pathogens Septoria nodorum or Septoria tritici, as well as antibodies which may be produced to act against these antigens. The antibodies which may be produced are those in the process described in EP 0472498 A1.

The said antibodies may be monoclonal or polyclonal antibodies, as selected, which can be produced by processes known perse, as are described e.g. in Ivan Roit, Jonathan Brostoff, David, K. Male, Lehrbuch der Immunologie, Georg Thieme Verlag, Stuttgart, 1991, 335f. or in R. T. V. Fox, 1993: Principles of Diagnostic Techniques in Plant Pathology, CAB, UK, pp 129-152.

Preferred indicator substances which are characteristic of certain plant pathogens may be selected from the group of receptors, ligands, proteins, antigens, oligonucleotides, strands of RNA or DNA, circular RNA, enzymes, enzyme substrates, enzyme cofactors, inhibitors or lectins.

Preferred indicator substances which are characteristic of certain plant pathogens may be selected from the group of cellulases, chitinases, PR proteins (pathogenesis related proteins) cutinases, amylases, pectinases, fatty acids or quinones.

Various specific binding partners can be applied to the surface of a waveguiding region, the physical separation thereof within each waveguiding region being unimportant. They can for example be present thereon in the form of a random mixture. This is advantageous when analytes having different emission wavelengths are to be determined simultaneously by way of a coupling-out grating.

The specific binding partners on the surface of each waveguiding region are preferably physically separate from one another.

The specific binding partners may be immobilised at various sites on the waveguiding regions, for example by photochemical crosslinking, as described in WO 94/27 137. Another method comprises the dropwise application of the specific binding partners that are to be immobilised, using a multiple-pipette head. This can also be effected using a modified inkjet printing head with piezoelectric actuators. This has the advantage that the method can be carried out rapidly and that very small amounts can be used. This is a precondition for the production of thin strips or other finely structured geometric patterns.

Another preferred method for the physically separate immobilisation of the specific binding partners on the waveguiding regions that is very simple to carry out is based on the use of a flow cell, it being possible for the separation to be effected in the flow cell, either mechanically in the form of dividing bars or fluidically in the case of laminar flow. In that method, the geometric arrangement of the part streams supplying the binding partners corresponds substantially to the arrangement of the waveguiding regions on the sensor platform. This method of immobilisation using a flow cell is advantageous especially when the specific binding partners are to be embedded in an environment that is stable only in the fluid medium, as is the case for example with lipid-membrane-bound receptors.

In particular, it is possible in this way to deposit specific binding partners that are covalently bonded to gold colloids, in the same manner as described above for the production of non-waveguiding regions. In order to obtain waveguiding in the immobilisation regions, it is necessary to use gold colloids of very small diameters of less than 10 nm and especially of less than 5 nm.

A further method that is likewise simple to carry out is based on stamping the surface with the specific binding partners, or with specific binding partners bonded to metals, in a manner analogous to that described above for the production of non-waveguiding regions.

A preferred metal is gold.

Preferred physically separate patterns are strips, rectangles, circles, ellipses or cross-hatches patterns.

Preference is given especially to a modified sensor platform which is characterised in that only one specific binding partner is arranged on the surface of each waveguiding region.

Another preferred embodiment of the modified sensor platform is obtained if an adhesion-promoting layer is located between the waveguiding regions and the immobilised specific binding partners.

The thickness of the adhesion-promoting layer is preferably equal to or less than 50 nm, especially less than 20 nm.

It is possible, furthermore, for adhesion-promoting layers to be applied selectively only in the waveguiding regions or to be passivated in the non-waveguiding regions, for example by means of photochemical activation or using wet-chemical methods, such as a multiple-pipette head, inkjet printers, flow cells with mechanical or fluidic separation of the streams, deposition of colloids or stamping of the surface. The methods have already been described above for the direct immobilisation of the specific recognition elements on an optionally chemically modified or functionalised surface.

The selective immobilisation of the specific recognition elements exclusively on the waveguiding regions, either directly or by way of adhesion-promoting layers, can, when using a sample cell that covers both the waveguiding and the non-waveguiding regions, lead to an increase in the sensitivity of the detection method, since the non-specific binding of the analytes in the regions not used for signal generation is reduced.

The preferences described hereinbefore for the sensor platform apply likewise to the modified sensor platform.

The modified sensor platform is preferably fully or partially regenerable and can be used several times. Under suitable conditions, for example at low pH, at elevated temperature, using organic solvents, or using so-called chaotropic reagents (salts), the affinity complexes can be selectively dissociated without substantially impairing the binding ability of the immobilised recognition elements. The precise conditions are greatly dependent upon the individual affinity system.

A specific form of luminescence detection in an assay consists in the immobilisation of the luminescent substances that are used for detection of the analyte directly on the surface of the waveguiding regions. These substances may be, for example, a plurality of luminophores bound to a protein which can thus be excited to luminescence on the surface of the waveguiding regions. If partners having affinity for the proteins are passed over that immobilised layer, the luminescence can be altered thereby and the quantity of partners having affinity can thus be determined. In particular, it is also possible for both partners of an affinity complex to be labelled with luminophores, in order for example to carry out determinations of concentration on the basis of the energy transfer between the two, for example in the form of luminescence extinction.

Another preferred embodiment of immobilisation for chemical or biochemical affinity assays consists in the immobilisation on the surface of the sensor platform of one or more specific binding partners as chemical or biochemical recognition elements for the analytes themselves or for one of the binding partners. The assays may consist of one or more stages in the course of which, in successive steps, one or more solutions containing specific binding partners for the recognition elements immobilised on the surface of the sensor platform can be passed over the surface of the sensor platform, the analytes being bound in one of the part steps. The analytes are detected by the binding of luminescently labelled participants in the affinity assay. The luminescence-labelled substances may be any one or more of the binding partners of the affinity assay, or an analogue of the analytes provided with a luminophore. The only precondition is that the presence of the analytes should lead selectively to a luminescence signal or selectively to a change in the luminescence signals.

In order to increase the chemically active sensor surface, it is also possible to immobilise the chemical or biochemical recognition elements on micro particles, so-called “beads”, which in turn can be fixed to the surface of the sensor platform by suitable methods. Prerequisites for the use of beads, which can consist of different materials, such as plastics, are that, firstly the interaction with the analyte takes place to a significant extent within the evanescent field of the waveguide, and secondly that the waveguiding properties are not significantly impaired. In principle, the recognition elements can be immobilised, for example, by

hydrophobic adsorption or covalent bonding directly on the waveguiding regions or after chemical modification of the surface, for example by silanisation or the application of a polymer layer. In addition, in order to facilitate the immobilisation of the recognition elements directly on the waveguide, a thin intermediate layer, for example consisting of SiO₂, can be applied as adhesion-promoting layer. The silanisation of glass and metal surfaces has been described comprehensively in literature, for example in Advances in Colloid and Interface Science 6, L. Boksányi, O. Liardon and E. Kováts, (1976) 95-137. Specific possible methods of carrying out the immobilisation have already been described hereinbefore.

Suitable recognition elements are, for example, antibodies for antigens, binding proteins such as protein A and G for immunoglobulins, biological and chemical receptors for ligands, chelators for “histidine-tag components”, for example histidine-labelled proteins, oligo-nucleotides and single strands of RNA or DNA for their complementary strands, avidin for biotin, enzymes for enzyme substrates, enzyme cofactors or inhibitors, or lectins for carbohydrates. Which of the relevant affinity partners is immobilised on the surface of the sensor platform depends on the architecture of the assay. The recognition elements may be natural or may be produced or synthesised by means of genetic engineering or biotechnology.

The expression antibodies includes both polyclonal and monoclonal antibodies, and fragments thereof.

The expressions ‘recognition element’ and ‘specific binding partner’ are used synonymously.

The assays themselves may be either one-step complexing processes, for example competitive assays, or multi-step processes, for example sandwich assays.

In the simplest example of a competitive assay, the sample, which comprises the analyte in unknown concentration and a known amount of a compound that is identical apart from being luminescence-labelled, is brought into contact with the surface of the sensor platform, where the luminescence-labelled and untabelled molecules compete for the binding sites on their immobilised recognition elements. In this assay configuration, a maximum luminescence signal is obtained when the sample contains no analyte. As the concentration of the substance to be detected increases, the observable luminescence signals decrease.

In a competitive immunoassay, the recognition element immobilised on the surface of the sensor platform does not have to be the antibody, but may alternatively be the antigen. It is generally a matter of choice in chemical or biochemical affinity assays which of the partners is immobilised. This is one of the principal advantages of assays based on luminescence over methods such as surface plasmon resonance or interferometry, which rely on a change in the adsorbed mass in the evanescent field of the waveguiding region.

Furthermore, the competition in the case of competitive assays need not be limited to binding sites on the surface of the sensor platform. For example, a known amount of an antigen can be immobilised on the surface of the sensor platform and then brought into contact with the sample which comprises as analyte an unknown amount, which is to be detected, of the same antigen and also luminescence-labelled antibodies. In this case, the competition to bind the antibodies takes place between antigens immobilised on the surface and antigens in solution.

A preferred embodiment is described in application examples B. Septoria nodorum or tritici spores are bound by the polyclonal antibodies to Septoria nodorum or Septoria tritici immobilised on the sensor plate. Then, the sample is brought into contact with the surface which comprises as analyte an unknown amount, to be detected, of the same antigen of Septoria nodorum spores or Septoria tritici spores, as well as luminescence-labelled antibodies to Septoria nodorum or Septoria tritici, In this case, there is competition between Septoria nodorum spores or Septoria tritici spores immobilised on the surface and in solution for binding of the Septoria nodorum antibodies or Septoria tritici antibodies.

The simplest example of a multi-step assay is a sandwich immunoassay in which a primary antibody is immobilised on the surface of the sensor platform. The binding of the antigen to be detected and of the luminescence-labelled secondary antibody used for the detection to a second epitope of the antigen can be effected either by contact with, in succession, the solution containing the antigen and a second solution containing the luminescence-labelled antibody, or after previously bringing the two solutions together so that finally the part-complex consisting of antigen and luminescence-labelled antibody is bound.

An especially preferred embodiment is a multi-step sandwich immunoassay, in which the primary antibody and the luminescence-labelled antibody are antibodies which are directed against Septoria nodorum antigens or against Septoria tritici antigens, and the antigen to be examined is Septoria nodorum antigen or Septoria tritici antigen.

Affinity assays may also comprise further additional binding steps. For example, in the case of sandwich immunoassays, in a first step protein A can be immobilised on the surface of the sensor platform. The protein specifically binds immunoglobulins to its so-called Fc portion and these then serve as primary antibodies in a subsequent sandwich assay which can be carried out as described.

There are many other forms of affinity assay, for example using the known avidin-biotin affinity system.

Examples of forms of affinity are to be found in J. H. Rittenburg, Fundamentals of Immuno-assay; in Development and Application of Immunoassay for Food Analysis, J. H. Rittenburg (Ed.), Elsevier, Essex 1990, or in P. Tijssen, Practice and Theory of Enzyme Immunoassays, R. H. Burdon, P. H. van Knippenberg (Eds), Elsevier, Amsterdam 1985; U.S. Pat. No. 4,868,105.

A further subject of the invention is a method for the parallel determination or one or more luminescences using a sensor platform or modified sensor platform for the diagnosis or plant diseases, which method comprises bringing one or more liquid samples into contact with one or more waveguiding regions on the sensor platform, coupling excitation light into the waveguiding regions, causing it to pass through the waveguiding regions, thus exciting in parallel in the evanescent field the luminescent substances in the samples or the luminescent substances immobilised on the waveguiding regions and, using optoelectronic components, measuring the luminescences produced thereby.

The preferences described hereinbefore for the sensor platform and the modified sensor platform apply also to the method of diagnosing plant diseases.

Only substantially parallel light is suitable for luminescence excitation. Substantially parallel is understood within the context of this invention to mean a divergence of less than 5°. This means that the light may be slightly divergent or slightly convergent. The use of coherent light for the luminescence excitation is preferred, especially laser light having a wavelength of 300 to 1100 nm, especially 450 to 850 nm, most particularly 480 to 700 nm.

Examples of lasers that may be used are dye lasers, gas lasers, solid state lasers and semiconductor lasers. If necessary, the emission wavelength can also be doubled by means of non-linear crystal optics. Using optical elements, the beam can also be focused further, polarised or attenuated by means of grey filters. Especially suitable lasers are argon/ion lasers and helium/neon lasers which emit at wavelengths of between 457 nm and 514 nm and between 543 nm and 633 nm respectively. Very especially suitable are diode lasers or frequency-doubled diode lasers of semiconductor material that emit at a fundamental wavelength of between 630 nm and 1100 nm, since, owing to their small dimensions and low power consumption, they allow substantial miniaturisation of the sensor system as a whole.

By “sample” is understood within the context of the present invention the entire solution to be analysed, which may contain a substance to be detected—the analyte. The detection may be effected in a one-step or multiple-step assay, during the course of which the surface of the sensor platform is brought into contact with one or more solutions. At least one of the solutions used contains a luminescent substance which can be detected according to the invention. If a luminescent substance has already been adsorbed onto the waveguiding region, the sample may also be free of luminescent constituents. The sample may contain further constituents, such as pH buffers, salts, acids, bases, surfactants, viscosity-influencing additives or dyes. In particular, a physiological saline solution can be used as solvent. If the luminescent portion is itself liquid, the addition of a solvent can be omitted. In that case, the content of luminescent substance in the sample may be up to 100%.

The sample may also be a biological medium, such as solutions of extracts from natural or synthetic media, such as soils or parts of plants, liquors from biological processes or plant extracts. Soil extracts are especially important for the diagnosis of soil-borne incidents.

The sample may be used either undiluted or with added solvent.

Suitable solvents are water, aqueous buffer solutions and protein solutions and organic solvents. Suitable organic solvents are alcohols, ketones, esters and aliphatic hydrocarbons. Preference is given to the use of water, aqueous buffers or a mixture of water with a miscible organic solvent.

However, the sample may also comprise constituents that are not soluble in the solvent, such as plant cell constituents, pigment particles, dispersants and natural and synthetic oligomers or polymers. The sample is then in the form of an optically opaque dispersion or emulsion.

Functionalised luminescent dyes having a luminescence of a wavelength in the range of 330 nm to 1000 nm may be used as luminescent compounds, for example rhodamines, fluorescein derivatives, NN382 (C₄₅H₄₈N₃O₁₃S₅Na₃), coumarin derivatives, distyryl biphenyls, stilbene derivatives, phthalocyanines, naphthalocyanines, polypyridyl/ruthenium complexes such as tris(2,2′-bipyridyl)ruthenium chloride, tris(1,10-phenanthroline)ruthenium chloride, tris(4,7-diphenyl-1,10-phenanthroline)ruthenium chloride and polypyridy/phenazine/ruthenium complexes, platinum/porphyrin complexes such as octaethyl-platinum-porphyrin, long-lived europium and terbium complexes or cyanine dyes. Dyes having absorption and emission wavelengths in the range of about 670 nm are not suitable for analyses in plant extracts which contain chlorophyll.

Very especially suitable are dyes, such as fluorescein derivatives, which contain functional groups by means of which they can be covalently bonded, for example fluorescein isothio-cyanate.

Also very suitable are the functional fluorescent dyes that are commercially available from the company LiCor, Lincoln, Nebr., USA, for example NN382 (C₄₅H₄₈N₃O₁₃Na₃), which are described for example in K. Behrmann, E. Birckner, E. Fanghaenel, J. Prakt. Chem. 326, 1034 (1984).

The preferred luminescence is fluorescence.

The use of different fluorescent dyes that can all be excited by light of the same wavelength, but have different emission wavelengths, may be advantageous, especially when using coupling-out gratings.

The luminescent dyes used may also be chemically bonded to polymers or to one of the binding partners in biochemical affinity systems, for example antibodies or antibody fragments, antigens, proteins, peptides, receptors or their ligands, hormones or hormone receptors, oligo-nucleotides, DNA and RNA strands, DNA or RNA analogues, binding proteins, such as protein A and G, avidin or biotin, enzymes, enzyme cofactors or inhibitors, lectins or carbohydrates. The use of the last-mentioned covalent luminescence labelling is preferred for reversible or irreversible (bio)chemical affinity assays. It is also possible to use luminescence-labelled steroids, lipids and chelators. In the case especially of hybridisation assays with DNA strands or oligonucleotides, intercalating luminescent dyes are also especially suitable, especially when—like various ruthenium complexes—they exhibit enhanced luminescence when intercalated. When these luminescence-labelled compounds are brought into contact with their affinity partners immobilised on the surface of the sensor platform, their binding can be readily quantitatively determined using the measured luminescence intensity. Equally, it is possible to effect a quantitative determination of the analytes by measuring the change in luminescence when the sample interacts with the luminophores, for example in the form of luminescence extinction by oxygen or luminescence enhancement resulting from conformation changes in proteins.

In the method according to the invention, the samples can be both brought into contact with the waveguiding regions when stationary, and passed over them continuously, it being possible for the circulation to be open or closed.

A further important form of application of the method is based on the one hand on limiting the generation of signals—in the case of backcoupling, this applies also to signal detection—to the evanescent field of the waveguide, and on the other hand on the reversibility of the affinity complex formation as an equilibrium process. Using suitable flow rates in a throughtlow system, the binding or desorption, i.e. dissociation, of bound, luminescence-labelled affinity partners in the evanescent field can be followed in real time. The method is therefore suitable for kinetic studies for determining different association or dissociation constants or for displacement assays.

The evanescently excited luminescence can be detected by known methods. Those suitable are photodiodes, photocells, photomultipliers, CCD cameras and detector arrays, such as CCD rows and CCD arrays. The luminescence can be projected onto the latter by means of optical elements, such as mirrors, prisms, lenses, Fresnel lenses and graded-index lenses, it being possible for the elements to be arranged individually or in the form of arrays. In order to select the emission wavelength, known elements, such as filters, prisms, monochromators, dichroic mirrors and diffraction gratings can be used.

The use of detector arrays arranged in the immediate vicinity of the sensor platform is advantageously, especially when a relatively large number of physically separate specific binding partners is present. Optical elements for separating excitation and luminescence light, such as holographic or interference filters, are advantageously arranged between the sensor platform and the detector array.

One embodiment of the method consists in detecting the isotropically radiated, evanescently excited luminescence.

In another embodiment of the method, the evanescently excited luminescence backcoupled into the waveguiding region is detected at an edge of the sensor platform or via a coupling-out grating. The intensity of the backcoupled luminescence is surprisingly high, with the result that very good sensitivity can likewise be achieved using this procedure.

In another form of the method, both the evanescently excited, isotropically radiated luminescence and the luminescence backcoupled into the waveguide are detected independently of one another but simultaneously. Owing to the different selectivity of these two luminescence detection methods, this selectivity being a function of the distance between the luminophores and the waveguiding region, this embodiment can be used to obtain additional information relating to the physical distribution of the luminophores. This also makes it possible to distinguish between photochemical bleaching of the luminophores and dissociation of the affinity complexes carrying the luminophores.

Another advantage of the method is that, in addition to the detection of luminescence, the absorption of the excitation light radiated in can be determined simultaneously. Compared with multimodal waveguides of fibre optic or planar construction, in this case a substantially better signal/noise ratio is achieved. Luminescence extinction effects can be detected with great sensitivity by means of the simultaneous measurement of luminescence and absorption.

The method can be carried out by radiating in the excitation light in continuous wave (cw) operation, i.e. the excitation is effected with light of an intensity that is constant over time.

However, the method can also be carried out by radiating in the excitation light in the form of a timed pulse having a pulse length of, for example, from one picosecond to 100 seconds and detecting the luminescence in a time-resolved manner—in the case of short pulse lengths—or at intervals from seconds to minutes. This method is especially advantageous if for example the rate of formation of a bond is to be followed analytically or the reduction in a luminescence signal resulting from photochemical bleaching is to be prevented using short exposure times. Furthermore, the use of suitably short pulse lengths and suitable time resolution of the detection make it possible to discriminate between scattered light, Raman emission and short-lived luminescence of any undesired luminescent constituents of the sample and of the sensor material that may be present, and the luminescence of the labelling molecule, which in this case is as long-lived as possible, since the emission of the analyte is detected only once the short-lived radiation has decayed. In addition, time-resolved luminescence detection after pulsed excitation, and likewise, modulated excitation and detection, allows investigation of the influence of the binding of the analyte on molecular luminescence decay behaviour. The molecular luminescence decay time can be used, alongside specific analyte recognition by the immobilised recognition elements and physical limitation of the generation of signals to the evanescent field of the waveguide, as a further selectivity criterion.

The method can also be carried out by radiating in the excitation light in an intensity modulated manner, at one or more frequencies, and detecting the resulting phase shift and modulation of the luminescence of the sample.

Parallel coupling of excitation light into a plurality of waveguiding regions can be carried out in several ways:

a) a plurality of laser light sources are used;

b) the beam from a laser light source is broadened using known suitable optical components, so that it covers a plurality of waveguiding regions and coupling-in gratings;

c) the beam from a laser light source is split using diffractive or holographically optical elements into a plurality of individual beams which are then coupled into the waveguiding regions via the gratings, or

d) an array of solid state lasers is used.

An advantageous procedure is also obtained by using a controllable deflecting mirror which can be used for coupling into or out of the waveguiding regions with a time delay. Alternatively, the sensor platform can be suitably displaced.

Another preferred method consists in exciting the luminescences with various laser light sources of identical or different wavelengths.

Preference is given especially to the use of a single row of diode lasers (laser array) for the excitation of the luminescences. These components have the special advantage that they are very compact and economical to produce, and the individual laser diodes can be individually controlled.

The preferences described for the sensor platform also apply in the case of the fluorescence detection method.

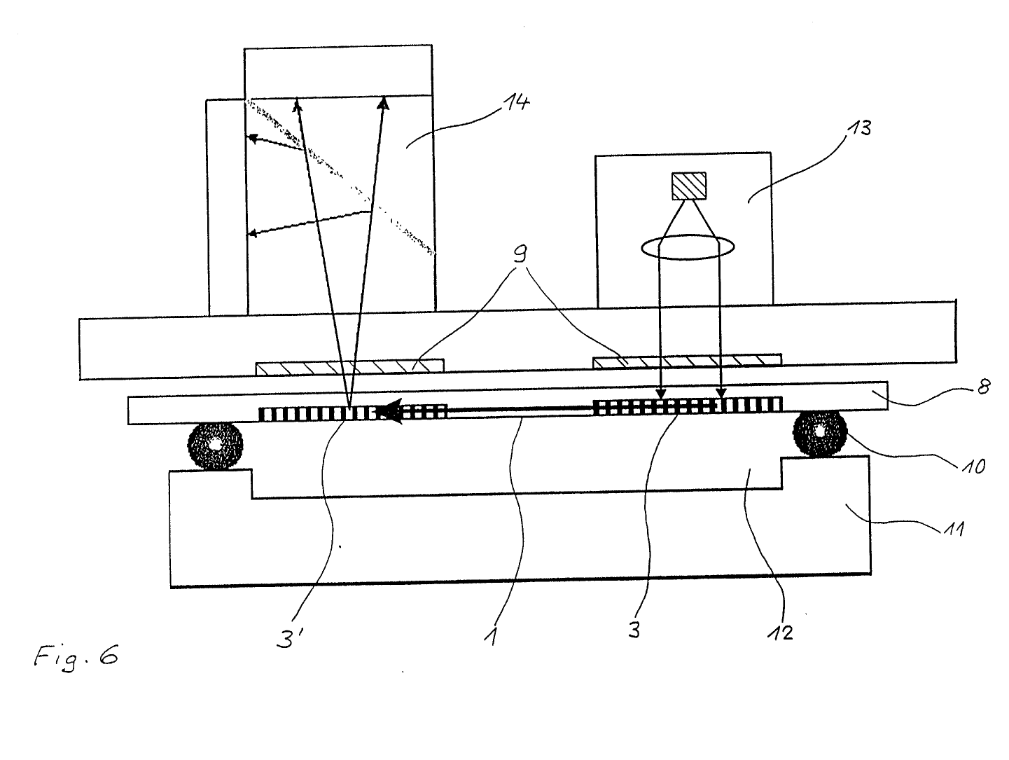

FIG. 6 is a schematic representation of a possible overall construction. Reference numerals 1 and 3 are as defined hereinbefore and other reference numerals are as follows:

8 sensor platform

9 filters

10 seal

11 throughflow cell

12 sample space

13 excitation optics

14 detection optics/electronics

The excitation light, for example from a diode laser 13, is coupled via a first grating 3 into a waveguiding region 1 of the sensor platform 8. On the underside of the sensor platform 8 and pressed tightly against the sensor platform is a throughflow cell 11. The solutions required for the assay are flushed through the space 12 in the throughflow cell 11, which may have one or more inlet openings and one or more outlet openings. The fluorescence of a binding partner is detected at the detector 14 onto which the fluorescence light backcoupled evanescently into the waveguiding region is coupled out via a second grating 3. The filters 9 serve to filter out scattered light.

The method is preferably used for analysing samples such as surface water, soil or plant extracts, and liquors from biological or synthetic processes.

The present invention also relates to the use of the sensor platform or modified sensor platform according to the invention for the quantitative determination of biochemical substances in affinity sensing, in the diagnosis of plant diseases.

Since signal generation and detection are limited to the chemical or biochemical recognition surface on the waveguide, and disturbance signals from the medium are discriminated, the binding of substances to the immobilised recognition elements can be followed in real time. The use of the method according to the invention in affinity screening or in displacement assays, especially in the diagnosis of plant diseases, by means of the direct determination of association and dissociation rates in a throughflow system at suitable flow rates, is therefore possible also.

The present invention also includes

a) the use of the sensor platform according to the invention or modified sensor platform according to the invention in processes for the diagnosis of plant diseases.

b) the use of the sensor platform according to the invention or modified sensor platform according to the invention in analytical processes for the diagnosis of plant diseases, preferably for the qualitative or quantitative determination of biochemical substances in affinity sensing.

c) the use of the sensor platform according to the invention or modified sensor platform according to the invention in an assay.

The assays in question may be assays with a one-step complexing process or a multi-step process.

Preference is given to the use of the sensor platform according to the invention or modified sensor platform according to the invention in sandwich assays, most preferably sandwich immuno-assays.

Particularly preferred is the use of the sensor platform according to the invention or modified sensor platform according to the invention in an assay in which a primary antibody is immobilised on the surface of the sensor platform, and binding of the antigen to be detected and of the luminescence-labelled secondary antibody used for the detection to a second epitope of the antigen can be effected by contact with, in succession, the solution containing the antigen and a second solution containing the luminescence-labelled antibody.

Preference is given to the use of the sensor platform according to the invention or modified sensor platform according to the invention in a sandwich immuno-assay in which a primary antibody is immobilised on the surface of the sensor platform, and binding of the antigen to be detected and of the luminescence-labelled secondary antibody used for the detection to a second epitope of the antigen is effected by previously bringing the two solutions together so that finally the part-complex consisting of antigen and luminescence-labelled antibody is bound.

Preference is given to the use of the sensor platform according to the invention or modified sensor platform according to the invention in a competitive assay.

Particular preference is given to the use of the sensor platform according to the invention or modified sensor platform according to the invention in a competitive immuno-assay.

Particular preference is given to the use of the sensor platform according to the invention or modified sensor platform according to the invention in a competitive assay in which competition is restricted to the binding sites on the surface of the sensor platform.

Preference is given to the use of the sensor platform or modified sensor platform in a competitive assay in which competition takes place between antigens that are immobilised on the surface of the sensor platform and those in solution for binding of the antibodies in solution.

Particularly preferred is the use of the sensor platform according to the invention or modified sensor platform according to the invention in a competitive assay in which a known amount of an antigen is immobilised on the surface of the sensor platform and then brought into contact with the sample which comprises as analyte an unknown amount, which is to be detected, of the same antigen and also luminescence-labelled antibodies. Preference is given to the use of the sensor platform according to the invention or modified sensor platform according to the invention in an assay in which Septoria nodorum or Septoria tritici antigens are bound by the antibodies to Septoria nodorum or the antibodies to Septoria tritici, which are immobilised on the sensor plate, and subsequently the sample is brought into contact with the surface which comprises as analyte an unknown amount to be detected of the same antigen of Septoria nodorum spores or Septoria tritici, as well as luminescence-labelled antibodies to Septoria nodorum or Septoria tritici.

d) the use of the sensor platform according to the invention or modified sensor platform according to the invention for the quantitative determination of antibodies or antigens, proteins, receptors or ligands, chelators or “histidine-tag components”, oligonucleotides, DNA or RNA strands, circular RNA, DNA or RNA analogues, enzymes, enzyme substrates, enzyme cofactors or inhibitors, lectins and carbohydrates, most preferably for the quantitative determination of antibodies or antigens.

e) the use of the sensor platform according to the invention or modified sensor platform according to the invention for the selective quantitative determination of luminescent components in optically opaque liquids, the optically opaque liquids being biological liquids such as samples from environmental analysis, for example surface water, dissolved earth extracts or dissolved plant extracts.

f) the use of the sensor platform according to the invention or modified sensor platform according to the invention for the detection of plant pathogens, whereby the above-mentioned definitions and preferences apply to plant pathogens.

g) the use of the sensor platform according to the invention or modified sensor platform according to the invention for the detection of indicator substances which are characteristic of certain plant pathogens, whereby the above-mentioned definitions and preferences apply to indicator substances.

h) the use of the biosensor according to the invention in processes for diagnosing plant diseases.

i) the use of the biosensor according to the invention in analytical processes for diagnosing plant diseases, preferably for the qualitative or quantitative determination of biochemical substances in affinity sensing.

j) the use of the biosensor according to the invention in an assay.

The assays in question may be assays with a one-step complexing process or a multi-step process.

Preference is given to the use of the biosensor according to the invention in sandwich assays, most preferably sandwich immuno-assays.

Particularly preferred is the use of the biosensor according to the invention in an assay in which a primary antibody is immobilised on the surface of the sensor platform, and binding of the antigen to be detected and of the luminescence-labelled secondary antibody used for the detection to a second epitope of the antigen can be effected by contact with, in succession, the solution containing the antigen and a second solution containing the luminescence-labelled antibody.

Preference is given to the use of the biosensor according to the invention in a sandwich immuno-assay in which a primary antibody is immobilised on the surface of the sensor platform, and binding of the antigen to be detected and of the luminescence-labelled secondary antibody used for the detection to a second epitope of the antigen is effected by previously bringing the two solutions together so that finally the part-complex consisting of antigen and luminescence-labelled antibody is bound.

Preference is given to the use of the biosensor according to the invention in a competitive assay.

Particular preference is given to the use of the biosensor according to the invention in a competitive immuno-assay.

Particular preference is given to the use of the biosensor according to the invention in a competitive assay in which competition is restricted to the binding sites on the surface of the sensor platform.

Preference is given to the use of the biosensor according to the invention in a competitive assay in which competition takes place between antigens that are immobilised on the surface of the sensor platform and those in solution for binding of the antibodies in solution. Particularly preferred is the use of the biosensor according to the invention in a competitive assay in which a known amount of an antigen is immobilised on the surface of the sensor platform and then brought into contact with the sample which comprises as analyte an unknown amount, which is to be detected, of the same antigen and also luminescence-labelled antibodies.

Preference is given to the use of the biosensor according to the invention in an assay in which Septoria nodorum or Septoria tritici antigens are bound by the antibodies to Septoria nodorum or the antibodies to Septoria tritici, which are immobilised on the sensor plate, and subsequently the sample is brought into contact with the surface which comprises as analyte an unknown amount, to be detected, of the same antigen of Septoria nodorum spores or Septoria tritici antigens, as well as luminescence-labelled antibodies to Septoria nodorum or Septoria tritici.

k) the use of the biosensor according to the invention for the quantitative determination of antibodies or antigens, proteins, receptors or ligands, chelators or “histidine-tag components”, oligonucleotides, DNA or RNA strands, circular RNA, DNA or RNA analogues, enzymes, enzyme substrates, enzyme cofactors or inhibitors, lectins and carbohydrates, most preferably for the quantitative determination of antibodies or antigens.

l) the use of the biosensor according to the invention for the selective quantitative determination of luminescent components in optically opaque liquids, the optically opaque liquid being biological liquids such as samples from environmental analysis, for example surface water, dissolved earth extracts or dissolved plant extracts.

m) the use of the biosensor according to the invention for the detection of plant pathogens, whereby the above-mentioned definitions and preferences apply to plant pathogens.

n) the use of the biosensor according to the invention for the—detection of indicator substances which are characteristic of certain plant pathogens, whereby the above-mentioned definitions and preferences apply to indicator substances.

The following examples illustrate the invention.

In all the following examples, the unit M of concentration denotes mol/I RT is room temperature, PAb-Septoria denotes polyclonal antibodies to Septoria.

### EXAMPLES A

### Production of various sensor platforms

### Example A1

### Production Using Masks in Vapour Deposition

A polycarbonate (PC) substrate is coated with TiO2 by means of vapour deposition (process: sputtering, deposition rate: 0.5 Å/s, thickness: 150 nm). Between the target and the substrate, in the immediate vicinity of the substrate, a mask is introduced. This is produced from aluminium, in which 6 strips 30 mm in length and 0.6 mm in width have been cut. The resulting 6 waveguiding regions (measuring regions) have a trapezoidal profile with a uniform thickness of 150 nm in the central region, which is 600 μm in width, and a layer thickness that decreases at the sides in the form of a gradient (shadowing). Coupled-in laser light is confined in the waveguiding region, since the effective refractive index is highest in the central region owing to the greatest layer thickness in that region.

### Example 2

### Production by Subsequent Division

The operation is carried out using an ArF excimer laser at 193 nm. The rectangular laser beam is concentrated using a cylindrical lens to a beam profile 200 μm wide and 20 mm long focused on the sensor platform. The sensor platform has a continuous 100 nm thick layer of Ta₂O₅. At an energy density above 1 J/cm² the entire layer is ablated with a single laser pulse (10 ns).

### Example A3

### Production by Subsequent Division

The operation is carried out using an Ar-ion laser at 488 nm. The round laser beam is concentrated using a microscope lens (40×) to a diameter of 4 μm focused on the waveguiding layer. The sensor platform has a continuous 100 nm thick layer of Ta₂O₅ and is located on a motor-controlled positioning element (Newport PM500). Under continuous laser irradiation, the platform is driven perpendicular to the beam at 100 mm/s. At an output of 700 mW, the entire waveguiding layer is ablated at the focus, with the result that two separate waveguiding regions are formed.

### Example A4

### Production by the Application of a Structured Absorbing Cover Layer by the Vacuum Method

5 parallel strips of a layer system of chromium/gold are vapour-deposited on the (continuous) metal oxide waveguide consisting of Ta₂O₅ (vapour-deposition installation: Balzers BAK 400); first 5 nm of Cr at 0.2 nm/s, then 45 nm of Au at 0.5 nm/s. The coupled-in models were interrupted at the absorbing layers.

### Example A5

### Production by the Application of a Structured Absorbing Cover Layer by the Aqueous Method

The surface of a metal oxide waveguide consisting of Ta₂O₅ is silanised with (mercapto-methyl)dimethylethoxysilane in the gas phase at 180° C. With the aid of a fine pipette, colloid solution A (GoldSol supplied by Aurion, average colloid diameter=28.9 nm, concentration: A₅₂₀≈1, aqueous solution) is applied to the modified surface in the form of droplets or strips and incubated for 1 hour. After the incubation, the surface is washed with water. Guided modal light is absorbed at the incubated sites. Downstream of the incubated sites, modal light is no longer present. The same applies in the case of protein A-covered Au colloid solution B (P-9785 supplied by Sigma, average diameter=18.4 nm, A₅₂₀≈5.5, in 50% glycerol, 0.15 M NaCl, 10 mM sodium phosphate, pH 7.4, 0.02% PEG 20, 0.02% sodium azide). The absorbing patterns on the waveguide surface are still intact even after flushing several times with water and with ethanol, which demonstrates the stability of the structures produced.

By the manual application of rows of microdrops (1 μl) of colloid solution A, continuous light-absorbing strips can be produced.

### Example A6

### Production by the Application of a Structured Absorbing Cover Layer by the Aqueous Method

The surface of a metal oxide waveguide consisting of TiO₂ is silanised with (mercaptomethyl)-dimethylethoxysilane in the gas phase at 40° C. Then a portion of the waveguide surface in front of and including the second coupling-out grating is incubated for 3 hours with colloid solution B (P-9785 supplied by Sigma, average diameter=18.4 nm, As2O=5.5, in 50% glycerol, 0.15 M NaCl, 10 mM sodium phosphate, pH 7.4, 0.02% PEG 20, 0.02% sodium azide). The wave propagation at the incubated sites is interrupted completely. The surface of the incubated site is examined using an atomic force microscope and the presence of colloids and the density of the gold particles anchored to the surface, that is necessary for the observed light absorption, are determined. The average separation of the particles is in the region of approx. 100 nm.

### Example A7

### Production by the Application of a Structured Absorbing Cover Layer by the Aqueous Method

The surface of a metal oxide waveguide consisting of Ta₂O₅ is silanised with (mercapto-methyl)dimethylethoxysilane (in the gas phase at 180° C.). The waveguide chip is connected to a throughflow cell having parallel, fluidically separate laminar part streams which allow up to five different streams of fluid to be passed in parallel adjacent to one another over the length of the waveguide surface via separate, individually addressable flow openings (1-5). The intention is to produce three waveguiding strips separated by two thinner strips of deposited Au colloids. The throughflow cell is charged at inlets 1, 3 and 5 with buffer (phosphate-buffered sodium chloride solution, pH 7.0) and at inlets 2 and 4 with Au colloid solution. A colloid solution, the surface of which is blocked with bovine serum albumin (BSA Gold Tracer supplied by Aurion, average colloid diameter=25 nm, OD₅₂₀≈2.0), is used. The flow rates selected (per channel) are: 0.167 ml/min for the buffer streams 1, 3 and 5, and 0.05 ml/min for the two colloid streams 2 and 4. This results in a width of approx. 1 mm for the colloid stream and approx. 3 mm for the buffer stream. The ratio of colloid stream width to buffer stream width can generally be freely selected via the ratio of the streams. The streams are applied for 20 mins. (corresponding to an amount of colloid of 1 ml per channel). After 20 minutes incubation, the waveguide chip is removed, washed with water and dried with a stream of nitrogen. Guided modal light is completely absorbed by the colloid-immobilised strips and results in three separate light-guiding modes of approx. 3 mm in width.

### APPLICATION EXAMPLES B

### Example B 1

### Detection of a Wheat Fungus Antigen (Septoria nodorum or Septoria tritici) Using a Sensor Platform Having a Single Waveguiding Region Covering the Whole Platform

B 1.1 Optical System

The light source used is a laser diode at λ=785 nm (Oz-Optics). With the assistance of an imaging system, it is adjusted to a beam spot with a diameter in the sensor plane of 0.4 mm vertical to the lines of the coupling grating and 2.5 mm parallel to the grating lines.

Adjustment of the coupling-in angle and positioning of the beam spot in respect of the grating edge is carried out by means of mechanical adjustment units.

The laser power on the sensor platform can be selected within the range P=0 . . . 3 mW. For the experiments described in the following to characterise the grating, P=1.2 mW was used, for the fluorescence measurements p=0.3 mW. By having rotatable polarising elements, linearly polarised light with TE- or TM-orientation can be coupled in as desired.

A throughflow cell is arranged on the upper side of the sensor platform. It is sealed against the sensor with O-rings, and the sample space of this cell is ca. 8 μl. Various solutions can be introduced into the cell using injection pumps and switch valves.

Excitation and detection are effected from the underside of the sensor platform.

Several measuring channels are available for detection. For the embodiment of an assay described in the following, the fluorescence which is excited in the evanescent field, but is reflected isotropically into the half space on the other side underneath the sensor platform, is recorded. This takes place in a measuring system as described in WO 95/33197.

To avoid spectral cross-talking in the detection of excitation light and emission light, interference filters are used in the excitation and emission light paths, in the emission path with a band pass 780 nm (30 nm band width, Omega Optical), in the emission path with a band pass 830 nm (40 nm band pass, Omega Optical). The fluorescence signals are recorded by a Single Photon Counting unit (Hamamatsu H6240-02-B1, with Photomultiplier R2949). The outgoing signals thereof can be transmitted to a conventional impulse counter (Hewlett Packard 53131 A). Si-diodes (UDT PIN 10 D) with a measuring amplifier (UDU 101 C) connected in series may be used as reference detectors.

B 1.2 Sensor Platform

The substrate used is polycarbonate, which is micro-structured in the following way with two gratings for coupling-in and coupling-out:

Coupling-in grating with period Λ₁=(370±2 nm), depth t₁=12.5 nm to 17.5 nm,

Coupling-out grating with period Λ₂=(580±3 nm), depth t₂=12.5 nm to 17.5 nm, both gratings with approximately sin⁴-shaped profile.

The gratings on the sensor platform are arranged with the following geometric sizes:

grating distance A=4 mm, grating width (vertically to lines) B₁=B₂=2 mm, grating height (parallel to lines) 4 mm, for dimensions of the sensor platform of 12×20 mm².

In order to suppress the polycarbonate intrinsic fluorescence, an intermediate layer of SiO2 with a refractive number n=1.46 and a thickness of t_{buffer}=(100±10) nm is applied to this substrate, and afterwards the high-refractive waveguiding layer of TiO₂ with the refractive number n_{film}=2.32 at λ=780 nm and the layer thickness t_{film}=(180±5) nm.

By means of the excitation light, for this grating-waveguide combination, the modes in the order of m=0 can be excited in the waveguide: for TEo coupling-in is effected at an angle of Θ=(−6.3±1.1)°, alternatively for TM₀ at an angle of Θ=(−20.4±3.3)°.

The fluorescence beam is coupled out with TE-polarisation at an angle range of Θ=29° . . . 38°, the excitation light at angles Θ>39°. A spectral range of λca. 800 nm . . . 830 nm corresponds to this angle range of fluorescence beam. For TM-polarisation, the coupling-out angle is Θ=15° . . . 23° for the fluorescence and Θ>24° for the excitation beam.

B 1.3. Solutions Employed

1) Buffer A:

8.8 g NaCl, 330 ml phosphate buffer pH 7, 50 ml methanol, 0.2 g sodium azide, 1 g BSA, 5 g Tween 20 [symbol] ad 1l H₂O.

2) Buffer B:

8.8 g NaCl, 330 ml phosphate buffer pH 7, 50 ml methanol, 0.2 g sodium azide [symbol] ad 1l H₂O

3) Regeneration buffer:

416.3 ml solution A, 463.7 ml HCl 0.1M, 120 ml isopropanol [symbol] pH 1.9

4) Solution A: glycine 0.1 M, NaCl 0.1 M

5) Standards (Septoria nodorum or Septoria tritici):

S1 10 million spores/ml extract from wheat leaves

S2 3 million spores/ml extract from wheat leaves

S3 1 million spores/ml extract from wheat leaves

S4 0.3 million spores/ml extract from wheat leaves

S5 0.1 million spores/ml extract from wheat leaves

S6 0.03 million spores/ml extract from wheat leaves

6) Buffer C:

40 mM Tris, 30 mM HCl, 150 mM NaCl, 0.1% BSA, 0.02% sodium azide [symbol] ad 1l H₂O, set at pH 7.7

B 1.4 Preparation of the Sensor Platform

The sensor platforms are silanised with (mercaptomethyl)dimethylethoxysilane (ABCR GmbH & Co., Karlsruhe) in gas phase (6 hours, 40° C., 0.2 mbar). After silanisation, the sensor platforms are incubated for 2 hours at room temperature with PAb-Septoria nodorum or PAb-Septoria tritici (0.3 mg/ml buffer B), washed with H₂O and then incubated for 1 hour at room temperature with Septoria nodorum spores or Septoria tritici spores (10 million spores/ml buffer B). The sensor platform is again washed with H₂O, blown dry with nitrogen and stored at −80° C. until measured.

Prior to the first measurement, the sensor platforms are incubated (20 mins; 0.5 ml/min) with buffer A in a throughflow cell in order to neutralise any free adsorption sites that may possibly be present on the surface.

B 1.5 Tracer Synthesis

300 μl of NN382 (C₄₅H₄₈N₃O₁₃S₅Na₃, LiCor, Lincoln, Nebr., USA, 1 mg/ml H₂O) are added to 700 μl of PAb-Septoria (0.86 mg/ml) in CO3²⁻/HCO3− buffer (pH 9.2). The reaction mixture is agitated for 2 hours at room temperature. Afterwards, the mixture is added to a PD-10 column (Pharmacia Biotech, Uppsala, Sweden), which was previously equilibrated with buffer B. The labelled antibody is eluted with the same buffer. By means of UV/VIS spectrometry, the concentration of the NN382-PAb-Septoria is set at 1×10⁻⁶ M, the solution is aliquoted and stored at −80° C. until measuring. The measuring concentration is respectively 2.5×10⁻⁹ M NN382-PAb-Septoria in buffer A.

B 1.6 Preparation of Extract from Wheat Leaves

The plant material is placed in a plastic bag and weighed. Then buffer C is added (1 ml per g plant material). The plant material in the plastic bag is then extracted using a macerator (Homex 6, Bioreba, Reinach).

B 1.7 Measuring Method

The measuring method consists of the following individual steps

5 minutes flushing with buffer A (0.5 ml/min.); recording of background signal

5 minutes supplying the sample (10 μl standard in 1.8 ml tracer, 0.25 ml/min)

2 minutes flushing with buffer A (0.5 ml/min.)

2 minutes supplying regeneration solution (0.5 ml/min.)

1 minute flushing with buffer A (0.5 ml/min.)

The specific signal is calculated from the difference in signal levels at t=12 mins and t=5 mins.

B 1.8 Results

The present assay is a competitive process, forming a sandwich complex consisting of the immobilised complex of PAb-Septoria nodorum or tritici and Septoria nodorum or tritici antigen, as well as the NN382-PAb-Septoria bound from the sample. Here, competition for the NN382-PAb-Septoria tracer takes place between the immobilised antigen and that found in the sample. A maximum fluorescence signal is produced at the lowest number of spores in the sample (S6).

| specific signal with S6 | background signal | signal noises |
|---|---|---|
| 40000 impulses | 2000 impulses | 100 impulses |
| per second | per second | per second |

Example B 2

### Parallel Detection of Two Wheat Fungus Antigens (Septoria nodorum and Septoria tritici) with Dfferent Recognition Elements Immobilised on 2 Physically Separate Waveguiding Regions

B 2.1 Optical Sensor Platform with Two Waveguiding Regions, Obtained According to Example A6

The sensor platform (metal oxide waveguide comprising TiO₂ with a surface of 12 mm×20 mm with identical parameters to those of example B 1.2) is silanised in gas phase with (mercapto-methyl)dimethylethoxysilane (ABCR GmbH & Co., KarLsruhe) (6 hours, 40°C., 0.2 mbar). Using an added fluid cell, in order to apply an absorbing cover layer in the region in which the waveguide is to be interrupted, the surface of the sensor platform is brought into contact with colloid solution B (P-9785 from Sigma, average diameter=18.4 nm, A₅₂₀≈5.5, in 50% glycerol, 0.15 M NaCL, 10 mM sodium phosphate, pH 7.4, 0.02% PEG 20, 0.02% sodium azide). The area of contact with the colloid solution comprises a rectangle of the dimensions 0.5 mm×10 mm, which extends beyond the coupling-in and coupling-out grating, and is localised in a central position of the grating height, so that 1.75 mm of the grating remains unchanged at the top and bottom. In the incubated region, the waveguide is completely interrupted.

B 2.2 Immobilisation Process

After flushing with water, the structured sensor platform is brought into contact with a fluid cell, which includes 2 separate flow channels at a distance of 0.5 mm. The dimensions of the two flow channels, with a depth of 0.2 mm, are respectively 1.5 mm height (parallel to the grating lines of the sensor platform) and 3.5 mm width (vertical to the grating lines of the sensor platform). The flow channels are arranged in relation to the sensor platform in such a way that the region of interruption of the waveguide lies between the two channels and at least the coupling-out grating of the sensor platform lies outside of the flow channel. In the region of channel 1, the sensor platform is incubated for 2 hours at room temperature with PAb-Septoria nodorum, and in the region of channel 2 with PAb-Septoria tritici (each with 0.3 mg/ml in buffer B). Afterwards, the two channels are washed with H₂O and subsequently incubated for 1 hour at room temperature—channel 1 with Septoria nodorum spores and channel 2 with Septoria tritici spores (respectively 10 million spores/ml buffer 8). The sensor platform is subsequently washed with H₂O, dried by blowing nitrogen through and stored at −80° C. until measuring.

B 2.3 Optical Structure

The light source used is a laser diode at λ=785 nm (Oz-Optics). With the assistance of an imaging system, it is adjusted to a beam spot with a diameter in the sensor plane of 0.4 mm vertical to the lines of the coupling grating and 4 mm parallel to the grating lines, so that the whole height of the grating is illuminated.

Adjustment of the coupling-in angle and positioning of the beam spot in respect of the grating edge is carried out by means of mechanical adjustment units. The laser power on the sensor platform can be selected within the range P=0 . . . 3 mW. By having rotatable polarising elements, linearly polarised light with TE- or TM-orientation can be coupled in as desired.

A throughflow cell with 2 channels is arranged on the upper side of the sensor platform in such a way that the separate channels respectively enclose the similarly separate waveguiding regions on which the different recognition elements have been immobilised. The sample volume of each channel is ca. 3 μl. Various solutions can be introduced into the cell using injection pumps and switch valves.

Excitation and detection are effected from below the sensor platform.

Several measuring channels are available for detection. For the example which is described here, the fluorescence which is excited in the evanescent field in the separate waveguiding regions, but is reflected isotropically into the half space on the other side below the sensor platform, is recorded. This takes place in a variation of the measuring system as described in WO 95/33197 for the simultaneous recording of signals from 2 adjacent waveguiding regions. To this end, the fluorescence light from the two separate sensor regions, which is reflected into the half space below the sensor platform, is collected by a glass fibre optics. The inlet cross-section of the glass fibre optics are designed so that cross-talking of the signals from the two sensor regions is avoided and at the same time maximum fluorescence is recorded. If required, the coupling-in efficiency into the glass fibres may be further increased through a combination with appropriate lenses.

The optical structure otherwise corresponds to the system described in example B 1.1, but now designed for 2 separate optical channels to detect fluorescence.

To avoid spectral cross-talking in the detection of excitation light and emission light, interference filters are used in the excitation and emission light paths, in the emission path with a band pass 780 nm (30 nm band width, Omega Optical), in the emission path with a band pass 830 nm (40 nm band pass, Omega Optical). The fluorescence signals from the two sensor regions are respectively recorded by a Single Photon Counting unit (Hamamatsu H6240-02-B1, with Photomultiplier R2949). The outgoing signals thereof can be transmitted to a conventional impulse counter (Hewlett Packard 53131 A). Si-diodes (UDT PIN 10 D) with a measuring amplifier (UDU 101 C) connected in series may be used as reference detectors.

B 2.4 Solutions Emploved:

1) Buffer A:

8.8 g NaCl, 330 ml phosphate buffer pH 7, 50 ml methanol, 0.2 g sodium azide, 1 g BSA, 5 g Tween 20 ad 1 l H₂O.

2) Buffer B:

8.8 g NaCl, 330 ml phosphate buffer pH 7, 50 ml methanol, 0.2 g sodium azide [symbol] ad 1 l H₂O

3) generation buffer:

416.3 ml solution A, 463.7 ml HCl 0.1M, 120 ml isopropanol [symbol] pH 1.9

4) Solution A: glycine 0.1 M, NaCi 0.1 M

5) Standards (Septoria nodorum or Septoria tritici):

S1 10 million spores/ml extract from wheat leaves

S2 3 million spores/ml extract from wheat leaves

S3 1 million spores/ml extract from wheat leaves

S4 0.3 million spores/ml extract from wheat leaves

S5 0.1 million spores/ml extract from wheat leaves

S6 0.03 million spores/ml extract from wheat leaves

6) Buffer C:

40 mM Tris, 30 mM HCl, 150 mM NaCl, 0.1% BSA, 0.02% sodium azide [symbol] ad 1 l H₂O, set at pH 0.7

B 2.5 Tracer Synthesis

300 μl of NN382 (C₄₅H₄₈N₃O₁₃S₅Na₃, LiCor, Lincoln, Nebr., USA, 1 mg/ml H₂O) are added to 700 μl of PAb-Septoria (0.86 mg/ml) in CO3²⁻/HCO3− buffer (pH 9.2). The reaction mixture is agitated for 2 hours at room temperature. Afterwards, the mixture is added to a PD-10 column (Pharmacia Biotech, Uppsala, Sweden), which was previously equilibrated with buffer B. The labelled antibody is eluted with the same buffer. By means of UVNIS spectrometry, the concentration of the NN382-PAb-Septoria is set at 1×10⁻⁶ M, the solution is aliquoted and stored at −80° C. until measuring. The measuring concentration is respectively 2.5×10⁻⁹ M NN382-PAb-Septoria in buffer A.

B 2.6 Preparation of Extract from Wheat Leaves

The plant material is placed in a plastic bag and weighed. Then buffer C is added (1 ml per g plant material). The plant material in the plastic bag is then extracted using a macerator (Homex 6, Bioreba, Reinach).

B 2.7 Measurinc Method

Prior to the first measurement, the two separate regions of the sensor platform are incubated (20 mins; 0.5 ml/min) with buffer A in a throughflow cell in order to neutralise any free adsorption sites that may possibly be present on the surface.

The measuring method with the simultaneous supply of two different analytes to the two physically separate sensor regions by means of the sample cell consisting of 2 flow channels comprises the following individual steps:

5 minutes flushing with buffer A (0.5 ml/min.) through both channels and recording of the background signal

5 minutes supplying the sample:

10 μl Septoria nodorum standard in 1.8 ml NN382-PAb-Septoria nodorum (2.5×10⁻⁹ M, 0.25 ml/min) through channel 1

10 μl Septoria tritici standard in 1.8 ml NN382-PAb-Septoria tritici (2.5×10⁻⁹ M, 0.25 ml/min) through channel 2

2 minutes flushing with buffer A (0.5 ml/min.) through both channels and recording of the fluorescence signal

2 minutes supplying regeneration solution (0.5 ml/min.) through both channels

1 minute flushing with buffer A (0.5 ml/min.) through both channels

The specific signal is calculated from the difference in signal levels at t=12 mins and t=5 mins.

### Example B 3

### Alternative Detection of Two Wheat Fungus Antigens (Septoria nodorum and Septoria tritici) with Different Recognition Elements Immobilised on 2 Physically Separate Regions

B 3.1 Immobilisation Process

After flushing with water, the sensor platform is brought into contact with a fluid cell, which includes 2 separate flow channels at a distance of 0.5 mm. The dimensions of the two flow channels, with a depth of 0.2 mm, are respectively 1.5 mm height (parallel to the grating lines of the sensor platform) and 3.5 mm width (vertical to the grating lines of the sensor platform). The flow channels are arranged in relation to the sensor platform in such a way that the flow channels are symmetrical to the coupling gratings of the sensor platform and at least the coupling-out grating of the sensor platform lies outside of the flow channels. In the region of channel 1, the sensor platform is incubated for 2 hours at room temperature with PAb-Septoria nodorum, and in the region of channel 2 with PAb-Septoria tritici (each with 0.3 mg/ml in buffer B). Afterwards, the two channels are washed with H₂O and subsequently incubated for 1 hour at room temperature—channel 1 with Septoria nodorum spores and channel 2 with Septoria tritici spores (respectively 10 million spores/ml buffer B). The sensor platform is subsequently washed with H₂O, dried by blowing nitrogen through and stored at −80° C. until measuring.

B 3.2 Optical Structure

The light source used is a laser diode at λ=785 nm (Oz-Optics), With the assistance of an imaging system, it is adjusted to a beam spot with a diameter in the sensor plane of 0.4 mm vertical to the lines of the coupling grating and 1.5 mm parallel to the grating lines, so that the height of the two flow channels of the sample cell pressed onto the sensor platform can each be completely illuminated.

Adjustment of the coupling-in angle and positioning of the beam spot in respect of the grating edge is carried out by means of mechanical adjustment units.

The laser power on the sensor platform can be selected within the range P=0 . . . 3 mW. By having rotatable polarising elements, linearly polarised light with TE- or TM-orientation can be coupled in as desired.

A throughflow cell with 2 channels is arranged on the upper side of the sensor platform in such a way that the separate channels respectively enclose the similarly separate regions on which the different recognition elements have been immobilised. The sample volume of each channel is ca. 3 μl. Various solutions can be introduced into the cell using injection pumps and switch valves.

Excitation and detection are effected from below the sensor platform.

Several measuring channels are available for detection. For the example which is described here, the fluorescence which is excited in the evanescent field in the separate waveguiding regions, but is reflected isotropically into the half space on the other side below the sensor platform, is recorded. This takes place in a measuring system as described in example B 1.1.

By having an additionally mounted, computer-controlled translation unit with translation parallel to the grating lines, the point at which the excitation light meets the coupling-in grating and this excites fluorescence in the separate sensor regions can be varied. In this way, it is possible to excite and detect alternating fluorescence signals (at time intervals of ca. 8 seconds) from the separate sensor regions.

B 3.3 Solutions Employed:

1) Buffer A:

8.8 g NaCl, 330 ml phosphate buffer pH 7, 50 ml methanol, 0.2 g sodium azide, 1 g BSA, 5 g Tween 20 ad 1 l H₂O.

2) Buffer B:

8.8 g NaCl, 330 ml phosphate buffer pH 7, 50 ml methanol, 0.2 g sodium azide [symbol] ad 1 l H₂O

3) Regeneration buffer:

416.3 ml solution A, 463.7 ml HCl 0.1M, 120 ml isopropanol [symbol] pH 1.9

4) Solution A: glycine 0.1 M, NaCl 0.1 M

5) Standards (Septoria nodorum or Septoria tritici):

S1 10 million spores/ml extract from wheat leaves

S2 3 million spores/ml extract from wheat leaves

S3 1 million spores/ml extract from wheat leaves

S4 0.3 million spores/ml extract from wheat leaves

S5 0.1 million spores/ml extract from wheat leaves

S6 0.03 million spores/ml extract from wheat leaves

6) Buffer C:

40 mM Tris, 30 mM HCl, 150 mM NaCl, 0.1% BSA, 0.02% sodium azide ad 1 l H₂O, set at pH 7.7

B 3.4 Tracer Synthesis

300 μl of NN382 (C₄₅H₄₈N₃O₁₃S₅Na₃, LiCor, Lincoln, Nebr., USA, 1 mg/ml H₂O) are added to 700pi of PAb-Septoria (0.86 mg/ml) in CO3²⁻/HCO3− buffer (pH 9.2). The reaction mixture is agitated for 2 hours at room temperature. Afterwards, the mixture is added to a PD-10 column (Pharmacia Biotech, Uppsala, Sweden), which was previously equilibrated with buffer B. The labelled antibody is eluted with the same buffer. By means of UVNIS spectrometry, the concentration of the NN382-PAb-Septoria is set at 1×10⁻⁶ M, the solution is aliquoted and stored at −80° C. until measuring. The measuring concentration is respectively 2.5×10⁻⁹ M NN382-PAb-Septoria in buffer A.

B 3.5 Preparation of Extract from Wheat Leaves

The plant material is placed in a plastic bag and weighed. Then buffer C is added (1 ml per g plant material). The plant material in the plastic bag is then extracted using a macerator (Homex 6, Bioreba, Reinach).

B 3.6 Measuring Method

Prior to the first measurement, the two separate regions of the sensor platform are incubated (20 mins; 0.5 ml/min) with buffer A in a throughflow cell in order to neutralise any free adsorption sites that may possibly be present on the surface.

The measuring method with the simultaneous supply of two different analytes to the two physically separate sensor regions by means of the sample cell consisting of 2 flow channels comprises the following individual steps:

5 minutes flushing with buffer A (0.5 ml/min.); through both channels and recording of the background signal

5 minutes supplying the sample:

10 μl Septoria nodorum standard in 1.8 ml NN382-PAb-Septoria nodorum (2.5×10⁻⁹ M, 0.25 ml/min) through channel 1

10 μl Septoria tritici standard in 1.8 ml NN382-PAb-Septoria tritici (2.5×10⁻⁹ M, 0.25 ml/min) through channel 2

2 minutes flushing with buffer A (0.5 ml/min.) through both channels and recording of the fluorescence signal

2 minutes supplying regeneration solution (0.5 ml/min.) through both channels

1 minute flushing with buffer A (0.5 ml/min.) through both channels

The signals from the two separate sensor regions are recorded alternately during the whole assay.

The specific signal is calculated from the difference in signal levels at t=12 mins and t=5 mins.

1. Sensor platform, characterised in that one or more specific binding partners are immobilised on the surface as chemical or biochemical recognition elements for one or more, identical or different plant pathogens to be evaluated.

2. Sensor platform according to claim 1, characterised in that the specific binding partners as chemical or biochemical recognition elements are specific for the plant pathogens to be evaluated, which are selected from the group of fungi, bacteria, viruses, viroids and phytoplasmoses.

3. Sensor platform according to claim 2, characterised in that the specific binding partners as chemical or biochemical recognition elements are specific for the fungi to be evaluated, which are selected from the division Myxomycota or Eumycota.

4. Sensor platform according to claim 2, characterised in that the specific binding partners as chemical or biochemical recognition elements are specific for the fungi to be evaluated, which are selected from the subdivisions of Mastigomycotina, Zycomycotina, Ascomycotina, Basidiomycotina or Deuteromycotina.

5. Sensor platform according to claim 2, characterised in that the specific binding partners as chemical or biochemical recognition elements are specific for the fungi to be evaluated, which are selected from the group of the genus Aphanomyces, Pythium, Phytophthora, Plasmopara, Bremia, Pseudoperonospora or Peronospora.

6. Sensor platform according to claim 2, characterised in that the specific binding partners as chemical or biochemical recognition elements are specific for the fungi to be evaluated, which are selected from the group of the genera Podosphaera, Sphaerotheca, Erysiphe, Uncinula, Nectria, Giberella (Fusarium), Glomerella, Claviceps, Sclerotinia, Cochliobolus, Leptosphaeria (Septoria), Pyrenophora, Venturia, Guignardia.

7. Sensor platform according to claim 2, characterised in that the specific binding partners as chemical or biochemical recognition elements are specific for the fungi to be evaluated, which are selected from the group of the genera Uromyces, Puccinia Hemileia, Ustilago, Tilletia, Typhula.

8. Sensor platform according to claim 2, characterised in that the specific binding partners as chemical or biochemical recognition elements are specific for the bacteria to be evaluated, which are selected from the group Agrobacterium, Spiroplasma, Clavibacter, Erwinia, Pseudomonas, Xanthomonas or Xylella.

9. Sensor platform according to claim 2, characterised in that the specific binding partners as chemical or biochemical recognition elements are specific for the viruses to be evaluated, which are selected from the group carla virus, clostero virus, cucumber mosaic virus, luteo virus, nepo vinus, potex virus, poty virus or tobacco mosaic virus from the group of phytoplasmoses.

10. Sensor platform according to claim 1, characterised in that the specific binding partners as chemical or biochemical recognition elements are specific for indicator substances which are characteristic of certain plant pathogens or the properties thereof.

11. Sensor platform according to claim 10, characterised in that the indicator substances which are characteristic of certain plant pathogens are selected from the group of receptors, ligands, proteins, antigens, oligonucleotides, strands of RNA or DNA, circular RNA, enzymes, enzyme substrates, enzyme cofactors, inhibitors or lectins.

12. Sensor platform according to claim 11, characterised in that the indicator substances which are characteristic of certain plant pathogens are selected from the group of cellulases, chitinases, PR proteins (pathogenesis related proteins) cutinases, amylases, pectinases, fatty acids or quinones.

13. Sensor platform according to claim 1, characterised in that the specific binding partners as chemical or biochemical recognition elements are selected from the groups of antibodies, antigens, binding proteins A, binding proteins G, receptors, ligands, oligonucleotides, single strand RNA, single strand DNA, avidin, biotin, enzymes, enzyme substrates, enzyme cofactors, enzyme inhibitors, lectins, carbohydrates.

14. Sensor platform according to claim 1, characterised in that the specific binding partners as chemical or biochemical recognition elements are antibodies or antigens.

15. Sensor platform according to claim 1-14, characterised in that signal generation is based on an optical transduction mechanism.

16. Sensor platform according to claim 15, characterised in that signal generation is based on interaction of one or more, identical or different plant pathogens to be evaluated with one or more specific binding partners as chemical or biochemical recognition elements in the evanescent field of a waveguide.

17. Sensor platform according to claim 16, characterised in that signal generation is based on the change in a luminescence signal due to the interaction of one or more, identical or different plant pathogens to be evaluated with one or more specific binding partners as chemical or biochemical recognition elements, which are immobilised on the sensor platform.

18. Sensor platform according to claim 1, characterised in that the sensor platform consists of one region on a substrate.

19. Sensor platform according to claim 1, characterised in that the sensor platform consists of at least two separate regions on a common substrate.

20. Sensor platform according to claim 1, characterised in that identical or different analytes are detected and quantified in parallel.

21. Sensor platform according to claim 1, characterised in that the sensor platform in question is based on a planar, dielectric, optical waveguide.

22. Sensor platform according to claim 1, characterised in that the sensor platform in question is a planar, dielectric, optical sensor platform, with which luminescence is evanescently excited and detected on the basis of a waveguide.

23. Sensor platform according to claim 1, characterised in that the sensor platform in question is a sensor platform based on at least two planar, separate, inorganic, dielectric waveguiding regions on a common substrate.

24. Sensor platform according to claim 23, characterised in that the sensor platform consists of a continuous substrate and a transparent, planar, inorganic, dielectric waveguiding layer, which is characterised in that
a) the transparent, inorganic, dielectric waveguiding layer is subdivided at least in the measuring region into at least 2 waveguiding regions, such that the effective refractive index in the regions in which the wave is guided is greater than in the surrounding regions, or such that the subdivision of the waveguiding layer is formed by a material on the surface that absorbs the coupled-in light;
b) the waveguiding regions are each provided with or have a common coupling-in grating, so that the direction of propagation of the wave vector is maintained after coupling-in and
c) where appropriate, the waveguiding regions are each provided with or have a common coupling-out grating.

25. Sensor platform according to claim 24, characterised in that the waveguiding regions are arranged in the form of parallel strips.

26. Sensor platform according to claim 24, characterised in that the individual waveguiding regions are arranged as multiple-detection regions on the substrate.

27. Sensor platform according to claim 24, characterised in that the substrate is glass, quartz or a transparent thermoplastic plastic.

28. Sensor platform according to claim 24, characterised in that the waveguiding regions consist of TiO₂, ZnO, Nb₂O₅, Ta₂O₅, HfO₂, or ZrO₂.

29. Sensor platform according to claim 24, characterised in that the thickness of the waveguiding regions is 40 to 300 nm.

30. Sensor platform according to claim 24, characterised in that
a) the transparent, planar, inorganic dielectric waveguiding regions on the sensor platform are divided from each other at least along the measuring section by a jump in refractive index of at least 0.6, and
b) each region has one or two separate grating couplers or all regions together have one or two common grating couplers, whereby
c) the transparent, planar, inorganic dielectric waveguiding regions have a thickness of 40 to 160 nm, the modulation depth of the gratings is 3 to 60 nm and the ratio of modulation depth to thickness is equal to or less than 0.5.

31. Sensor platform according to claim 1, characterised in that the specific binding partners on the surface of each waveguiding region are physically separate from one another.

32. Process for the production of the sensor platform according to claim 24, characterised in that the inorganic waveguiding material undergoes vapour deposition in a vacuum under a suitably constructed mask.

33. Process for the production of the sensor platform according to claim 1, characterised in that the dissolved specific binding partners are guided by a multi-channel throughflow cell over the separate waveguiding regions, whereby the multi-channel cell has fluidic or physical separation of the channels.

34. Process for the parallel determination of one or more luminescences using a sensor platform or a modified sensor platform according to one of claim 17 or 1, characterised in that one or more liquid samples are brought into contact with one or more waveguiding regions on the sensor platform, excitation light is coupled into the waveguiding regions, causing it to pass through the waveguiding regions, thus exciting in parallel in the evanescent field the luminescent substances in the samples or the luminescent substances immobilised on the waveguiding regions and, using optoelectronic components, the luminescences produced thereby are measured.

35. Process according to claim 34, characterised in that the sample to be examined is surface water, a soil or plant extract, or a liquor from a biological or synthetic process.

36. Biosensor for diagnosing plant diseases, which contains a sensor platform according one of claims 1-31 and an appropriate transducer arrangement.

37. Biosensor according to claim 36, characterised in that the transducer arrangement detects optical changes based on luminescence.

38. Process for diagnosing plant diseases, characterised in that the sample to be examined is analysed for the presence and quantity of plant pathogens using a biosensor.

39. Process for diagnosing plant diseases, characterised in that a biosensor according to one of claims 36 or 37 is used.

40. Process for diagnosing plant diseases, characterised in that the sample to be examined is examined for the presence of plant pathogens using a sensor platform according to one of claims 1-31.

41. Use of the sensor platform according to one of claims 1-31 in analytical processes for diagnosing plant diseases.

42. Use of the sensor platform according to one of claims 1-31 an assay.

43. Use of the sensor platform according to claim 42 in an assay, characterised in that the assay is a sandwich assay.

44. Use of the sensor platform according to claim 42 in an assay, characterised in that the assay is a competitive assay.

45. Use of the sensor platform according to one of claims 1-31 for detecting plant pathogens.

46. Use of a biosensor according to claim 37 for detecting plant pathogens.

47. Use of a biosensor according to claim 46, characterised in that the plant pathogens to be evaluated are selected from the group of fungi, bacteria, viruses, viroids and phytoplasmoses.

48. Use of a biosensor according to claim 47, characterised in that the fungi to be determined are selected from the division Myxomycota or Eumycota.
